SEQUENCE LISTING (65 sequences)

SEQ ID NO: 1 - DNA, 5413 bp, Artificial Sequence
  plasmid
tgtgagtttctgtgtaactgatatcgccatttttccaaaagtgatttttgggcatacgcgatatctggcgatagcgcttatatcgtttacgggggatggcgatagacgactttggtgacttgggcgattctgtgtgtcgcaaatatcgcagtttcgatataggtgacagacgatatgaggctatatcgccgatagaggcgacatcaagctggcacatggccaatgcatatcgatctatacattgaatcaatattggccattagccatattattcattggttatatagcataaatcaatattggctattggccattgcatacgttgtatccatatcataatatgtacatttatattggctcatgtccaacattaccgccatgttgacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagctcgtttagtgaaccgtcagatcgcctggagacgccatccacgctgttttgacctccatagaagacaccgggaccgatccagcctccgcggccgggaacggtgcattggaacgcggattccccgtgccaagagtgacgtaagtaccgcctatagagtctataggcccacccccttggcttcttatgcatgctatactgtttttggcttggggtctatacacccccgcttcctcatgttataggtgatggtatagcttagcctataggtgtgggttattgaccattattgaccactcccctattggtgacgatactttccattactaatccataacatggctctttgccacaactctctttattggctatatgccaatacactgtccttcagagactgacacggactctgtatttttacaggatggggtctcatttattatttacaaattcacatatacaacaccaccgtccccagtgcccgcagtttttattaaacataacgtgggatctccacgcgaatctcgggtacgtgttccggacatgggctcttctccggtagcggcggagcttctacatccgagccctgctcccatgcctccagcgactcatggtcgctcggcagctccttgctcctaacagtggaggccagacttaggcacagcacgatgcccaccaccaccagtgtgccgcacaaggccgtggcggtagggtatgtgtctgaaaatgagctcggggagcgggcttgcaccgctgacgcatttggaagacttaaggcagcggcagaagaagatgcaggcagctgagttgttgtgttctgataagagtcagaggtaactcccgttgcggtgctgttaacggtggagggcagtgtagtctgagcagtactcgttgctgccgcgcgcgccaccagacataatagctgacagactaacagactgttcctttccatgggtcttttctgcagtcaccgtccttgacacgaagcttgggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtagggaattctgcagatatccatcacactggcggccgctcgagcatgcatctagagggccctattctatagtgtcacctaaatgctagagctcgctgatcagcctcgactgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatggcttctgaggcggaaagaaccagctggggctcgaggggggatcgatccgtcgacctcgagagcttggcgtaatcatggtcatagctgtttcctgtgtgaaattgttatccgctcacaattccacacaacatacgagccggaagcataaagtgtaaagcctggggtgcctaatgagtgagctaactcacattaattgcgttgcgctcactgcccgctttccagtcgggaaacctgtcgtgccagctgcattaatgaatcggccaacgcgcggggagaggcggtttgcgtattgggcgctcttccgcttcctcgctcactgactcgctgcgctcggtcgttcggctgcggcgagcggtatcagctcactcaaaggcggtaatacggttatccacagaatcaggggataacgcaggaaagaacatgtgagcaaaaggccagcaaaaggccaggaaccgtaaaaaggccgcgttgctggcgtttttccataggctccgcccccctgacgagcatcacaaaaatcgacgctcaagtcagaggtggcgaaacccgacaggactataaagataccaggcgtttccccctggaagctccctcgtgcgctctcctgttccgaccctgccgcttaccggatacctgtccgcctttctcccttcgggaagcgtggcgctttctcatagctcacgctgtaggtatctcagttcggtgtaggtcgttcgctccaagctgggctgtgtgcacgaaccccccgttcagcccgaccgctgcgccttatccggtaactatcgtcttgagtccaacccggtaagacacgacttatcgccactggcagcagccactggtaacaggattagcagagcgaggtatgtaggcggtgctacagagttcttgaagtggtggcctaactacggctacactagaagaacagtatttggtatctgcgctctgctgaagccagttaccttcggaaaaagagttggtagctcttgatccggcaaacaaaccaccgctggtagcggtggtttttttgtttgcaagcagcagattacgcgcagaaaaaaaggatctcaagaagatcctttgatcttttctacggggtctgacgctcagtggaacgaaaactcacgttaagggattttggtcatgaacaataaaactgtctgcttacataaacagtaatacaaggggtgttatgagccatattcaacgggaaacgtcttgctcgaggccgcgattaaattccaacatggatgctgatttatatgggtataaatgggctcgcgataatgtcgggcaatcaggtgcgacaatctatcgattgtatgggaagcccgatgcgccagagttgtttctgaaacatggcaaaggtagcgttgccaatgatgttacagatgagatggtcagactaaactggctgacggaatttatgcctcttccgaccatcaagcattttatccgtactcctgatgatgcatggttactcaccactgcgatccccgggaaaacagcattccaggtattagaagaatatcctgattcaggtgaaaatattgttgatgcgctggcagtgttcctgcgccggttgcattcgattcctgtttgtaattgtccttttaacagcgatcgcgtatttcgtctcgctcaggcgcaatcacgaatgaataacggtttggttgatgcgagtgattttgatgacgagcgtaatggctggcctgttgaacaagtctggaaagaaatgcataagcttttgccattctcaccggattcagtcgtcactcatggtgatttctcacttgataaccttatttttgacgaggggaaattaataggttgtattgatgttggacgagtcggaatcgcagaccgataccaggatcttgccatcctatggaactgcctcggtgagttttctccttcattacagaaacggctttttcaaaaatatggtattgataatcctgatatgaataaattgcagtttcatttgatgctcgatgagtttttctaatcagaattggttaattggttgtaacactggcagagcattacgctga

SEQ ID NO: 2 - DNA, 1094 bp, Artificial Sequence
  plasmid
aagcttgggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtagggaattc

SEQ ID NO: 3 - protein, 281 aa, Hepatitis B virus
MQWNSTTFHQTLQDPRVRGLYFPAGGSSSGTVNPVLTTASPLSSIFSRIGDPALNMENITSGFLGPLLVLQAGFFLLTRILTIPQSLDSWWTSLNFLGGTTVCLGQNSQSPTSNHSPTSCPPTCPGYRWMCLRRFIIFLFILLLCLIFLLVLLDYQGMLPVCPLIPGSSTTSTGPCRTCMTTAQGTSMYPSCCCTKPSDGNCTCIPIPSSWAFGKFLWEWASARFSWLSLLVPFVQWFVGLSPTVWLSVIWMMWYWGPSLYSILSPFLPLLPIFFCLWVYI

SEQ ID NO: 4 - DNA, 1196 bp, Artificial Sequence
  HBsAg gene insert in MVA.HBS
gtaaaacgacggccagtttgcacggtaaggaagtagaatcataaagaacagtgacggatcccagcttgggccatgcagtggaattccacaaccttccaccaaactctgcaagatcccagagtgagaggcctgtatttccctgctggtggctccagttcaggaacagtaaaccctgttctgactactgcctctcccttatcgtcaatcttctcgaggattggggaccctgcgctgaacatggagaacatcacatcaggattcctaggaccccttctcgtgttacaggcggggtttttcttgttgacaagaatcctcacaataccgcagagtctagactcgtggtggacttctctcaattttctagggggaactaccgtgtgtcttggccaaaattcgcagtccccaacctccaatcactcaccaacctcttgtcctccaacttgtcctggttatcgctggatgtgtctgcggcgttttatcatcttcctcttcatcctgctgctatgcctcatcttcttgttggttcttctggactatcaaggtatgttgcccgtttgtcctctaattccaggatcctcaacaaccagcacgggaccatgccggacctgcatgactactgctcaaggaacctctatgtatccctcctgttgctgtaccaaaccttcggacggaaattgcacctgtattcccatcccatcatcctgggctttcggaaaattcctatgggagtgggcctcagcccgtttctcctggctcagtttactagtgccatttgttcagtggttcgtagggctttcccccactgtttggctttcagttatatggatgatgtggtattgggggccaagtctgtacagcatcttgagtccctttttaccgctgttaccaattttcttttgtctttgggtatacatttaaaccctaacaaaacaaagagatggggttactctctaaattttatgggttatgtcattggatgttatgggtccttgccacaagaacacatcatacaaaaaatcaaagaatgttttagaaaacttcctattaacaggcctattgattggaaagtatgtcaacgaattgtgggtcttttgggttttgctgccccttttacacaatgtggttatcctgcgttgatgcctttgtagggaattctgtgagcgtatggcaaacgaaggaacatggtcatagctgtt

SEQ ID NO: 5 - DNA, 1196 bp, Artificial Sequence; identical to SEQ ID NO: 4
  HBsAg gene insert in MVA.HBS

SEQ ID NO: 6 - DNA, 21 bp, Artificial Sequence
  oligonucleotide primer
tcctgcgttgatgcctttgta

SEQ ID NO: 7 - DNA, 26 bp, Artificial Sequence
  oligonucleotide primer
caattacagatttctccgtgataggt

SEQ ID NO: 8 - DNA, 23 bp, Artificial Sequence
  oligonucleotide primer
tcatttgcactttctggttcgta

SEQ ID NO: 9 - DNA, 42 bp, Artificial Sequence
  oligonucleotide primer
gtaaaacgacggccagtttgcacggtaaggaagtagaatcat

SEQ ID NO: 10 - DNA, 38 bp, Artificial Sequence
  oligonucleotide primer
aacagctatgaccatgttccttggtttgccatacgctc

SEQ ID NO: 11 - DNA, 17 bp, Artificial Sequence
  oligonucleotide primer
gtaaaacgacggccagt

SEQ ID NO: 12 - DNA, 24 bp, Artificial Sequence
  oligonucleotide primer
cacacaggaaacagctatgaccat

SEQ ID NO: 13 - DNA, 19 bp, Artificial Sequence
  oligonucleotide primer
ggtcctaggaatcctgatg

SEQ ID NO: 14 - DNA, 19 bp, Artificial Sequence
  oligonucleotide primer
ggatgttatgggtccttgc

SEQ ID NO: 15 - protein, 12 aa, Hepatitis B virus
IPQSLDSWWTSL

SEQ ID NO: 16 - protein, 15 aa, Hepatitis B virus
MQWNSTTFHQTLQDP

SEQ ID NO: 17 - protein, 15 aa, Hepatitis B virus
TFHQTLQDPRVRGLY

SEQ ID NO: 18 - protein, 15 aa, Hepatitis B virus
QDPRVRGLYFPAGGS

SEQ ID NO: 19 - protein, 15 aa, Hepatitis B virus
GLYFPAGGSSSGTVN

SEQ ID NO: 20 - protein, 15 aa, Hepatitis B virus
GGSSSGTVNPVLTTA

SEQ ID NO: 21 - protein, 15 aa, Hepatitis B virus
TVNPVLTTASPLSSI

SEQ ID NO: 22 - protein, 15 aa, Hepatitis B virus
TTASPLSSIFSRIGD

SEQ ID NO: 23 - protein, 15 aa, Hepatitis B virus
SSIFSRIGDPALNME

SEQ ID NO: 24 - protein, 15 aa, Hepatitis B virus
IGDPALNMENITSGF

SEQ ID NO: 25 - protein, 15 aa, Hepatitis B virus
NMENITSGFLGPLLV

SEQ ID NO: 26 - protein, 15 aa, Hepatitis B virus
SGFLGPLLVLQAGFF

SEQ ID NO: 27 - protein, 15 aa, Hepatitis B virus
LLVLQAGFFLLTRIL

SEQ ID NO: 28 - protein, 15 aa, Hepatitis B virus
GFFLLTRILTIPQSL

SEQ ID NO: 29 - protein, 15 aa, Hepatitis B virus
RILTIPQSLDSWWTS

SEQ ID NO: 30 - protein, 15 aa, Hepatitis B virus
QSLDSWWTSLNFLGG

SEQ ID NO: 31 - protein, 15 aa, Hepatitis B virus
WTSLNFLGGTTVCLG

SEQ ID NO: 32 - protein, 15 aa, Hepatitis B virus
LGGTTVCLGQNSQSP

SEQ ID NO: 33 - protein, 15 aa, Hepatitis B virus
CLGQNSQSPTSNHSP

SEQ ID NO: 34 - protein, 15 aa, Hepatitis B virus
QSPTSNHSPTSCPPT

SEQ ID NO: 35 - protein, 15 aa, Hepatitis B virus
HSPTSCPPTCPGYRW

SEQ ID NO: 36 - protein, 15 aa, Hepatitis B virus
PPTCPGYRWMCLRRF

SEQ ID NO: 37 - protein, 15 aa, Hepatitis B virus
YRWMCLRRFIIFLFI

SEQ ID NO: 38 - protein, 15 aa, Hepatitis B virus
RRFIIFLFILLLCLI

SEQ ID NO: 39 - protein, 15 aa, Hepatitis B virus
LFILLLCLIFLLVLL

SEQ ID NO: 40 - protein, 15 aa, Hepatitis B virus
CLIFLLVLLDYQGML

SEQ ID NO: 41 - protein, 15 aa, Hepatitis B virus
VLLDYQGMLPVCPLI

SEQ ID NO: 42 - protein, 15 aa, Hepatitis B virus
GMLPVCPLIPGSSTT

SEQ ID NO: 43 - protein, 15 aa, Hepatitis B virus
PLIPGSSTTSTGPCR

SEQ ID NO: 44 - protein, 15 aa, Hepatitis B virus
STTSTGPCRTCMTTA

SEQ ID NO: 45 - protein, 15 aa, Hepatitis B virus
PCRTCMTTAQGTSMY

SEQ ID NO: 46 - protein, 15 aa, Hepatitis B virus
TTAQGTSMYPSCCCT

SEQ ID NO: 47 - protein, 15 aa, Hepatitis B virus
SMYPSCCCTKPSDGN

SEQ ID NO: 48 - protein, 15 aa, Hepatitis B virus
CCTKPSDGNCTCIPI

SEQ ID NO: 49 - protein, 15 aa, Hepatitis B virus
DGNCTCIPIPSSWAF

SEQ ID NO: 50 - protein, 15 aa, Hepatitis B virus
IPIPSSWAFGKFLWE

SEQ ID NO: 51 - protein, 15 aa, Hepatitis B virus
WAFGKFLWEWASARF

SEQ ID NO: 52 - protein, 15 aa, Hepatitis B virus
LWEWASARFSWLSLL

SEQ ID NO: 53 - protein, 15 aa, Hepatitis B virus
ARFSWLSLLVPFVQW

SEQ ID NO: 54 - protein, 15 aa, Hepatitis B virus
SLLVPFVQWFVGLSP

SEQ ID NO: 55 - protein, 15 aa, Hepatitis B virus
VQWFVGLSPTVWLSV

SEQ ID NO: 56 - protein, 15 aa, Hepatitis B virus
LSPTVWLSVIWMMWY

SEQ ID NO: 57 - protein, 15 aa, Hepatitis B virus
LSVIWMMWYWGPSLY

SEQ ID NO: 58 - protein, 15 aa, Hepatitis B virus
MWYWGPSLYSILSPF

SEQ ID NO: 59 - protein, 15 aa, Hepatitis B virus
SLYSILSPFLPLLPI

SEQ ID NO: 60 - protein, 15 aa, Hepatitis B virus
SPFLPLLPIFFCLWV

SEQ ID NO: 61 - protein, 15 aa, Hepatitis B virus
FLPLLPIFFCLWVYI

SEQ ID NO: 62 - DNA, 6 bp, Artificial Sequence
  restriction endonuclease site
aaggtt

SEQ ID NO: 63 - DNA, 6 bp, Artificial Sequence
  restriction endonuclease site
atgcat

SEQ ID NO: 64 - DNA, 5 bp, Artificial Sequence
  restriction endonuclease site
aggtt

SEQ ID NO: 65 - DNA, 5 bp, Artificial Sequence
  restriction endonuclease site
tcgaa

(19) United States
(12) Patent Application Publication
(10) Pub. No.: US 2008/0267996 A1
(43) Pub. Date: Oct. 30, 2008

(54) Compositions for inducing an immune response against hepatitis B

(75) Inventors: Joerg Schneider, Barton (GB); James Chorlton, Gerrards Cross (GB); Gill Pearce, Maidenhead (GB); Nicola Jones, Windsor (GB); Dean Brown, Didcot (GB)

Correspondence Address: HAMILTON, BROOK, SMITH & REYNOLDS, P.C., 530 VIRGINIA ROAD, P.O. BOX 9133, CONCORD, MA, 01742-9133, US

(73) Assignee: Oxxon Therapeutics Ltd., Oxford (GB)

(21) Appl. No.: 11/986,294

(22) Filed: Nov. 20, 2007

Related U.S. Application Data
(63) Continuation of application No. PCT/GB2006/001902, filed on May 23, 2006.
(60) Provisional application No. 60/782,710, filed on Mar. 15, 2006.
(60) Provisional application No. 60/683,877, filed on May 23, 2005.

(30) Foreign Application Priority Data
Jul. 27, 2005 (GB) 0515439.8

(51) Int. Cl. A61K 39/29; C12N 15/33; A61K 31/7088; A61K 35/76; A61P 31/12; C12N 7/00; C12N 15/63; A61K 39/275

(52) U.S. Cl. 424/205.1; 424/227.1; 435/235.1; 424/93.2; 536/23.72; 435/320.1; 514/44

(57) ABSTRACT

The present invention is directed to a method of inducing an immune response against a hepatitis B antigen (e.g., an antigen from a hepatitis B virus) in a mammal, which comprises administering to the mammal a priming composition (e.g., a DNA plasmid), comprising a source of one or more epitopes of the hepatitis B target antigen; and a boosting composition, comprising a source of one or more eptiopes of the hepatitis B target antigen (e.g., a non-replication or replication-impaired poxvirus such as MVA), wherein at least one epitope of the boosting composition is identical to an epitope of the priming composition. The present invention also is directed to a method of inducing an immune response against a hepatitis B antigen (e.g., an antigen from a hepatitis B virus) in a mammal, which comprises administering to the mammal a priming composition (e.g., a DNA plasmid), comprising a source of one or more epitopes of the hepatitis B target antigen. In addition, the present invention is directed to compositions for use in the methods of the present invention.

### RELATED APPLICATIONS

This application is a continuation of International Application No. PCT/GB2006/001902, which designated the United States, was filed on May 23, 2006, was published in English, claims the benefit of U.S. Provisional Application Nos. 60/782,710, filed on Mar. 15, 2006 and 60/683,877, filed on May 23, 2005, and which claims priority to Great Britain Application No. 0515439.8, filed Jul. 27, 2005. The entire teachings of the above applications are incorporated herein by reference.

### BACKGROUND OF THE INVENTION

Hepatitis B is caused by a 42 nm double-stranded DNA virus that is the prototype member of the hepadnavirus family. There are more than 350 million carriers of the hepatitis B virus (HBV) world-wide, and chronic active hepatitis leads to cirrhosis and hepatocellular carcinoma in approximately one quarter of these individuals. A major problem in HBV immunotherapy has been the identification of a means of inducing a sufficiently strong immune response in individuals with chronic hepatitis B. A number of different immunisation strategies have failed to generate clinically-effective immune responses against the infection in humans. There is a clear need for the development of improved methods for inducing an immune response to hepatitis B infection in an individual in need thereof.

### SUMMARY OF THE INVENTION

The present invention encompasses a method of inducing an immune response against a (one or more) hepatitis B antigen (e.g., an antigen from the hepatitis B virus) in a mammal (e.g. , human), which comprises administering to the mammal a priming composition (e.g., a DNA plasmid) comprising a source of one or more epitopes of the hepatitis B antigen; and a boosting composition comprising a source of one or more epitopes of the hepatitis B antigen (e.g., a non-replication or replication-impaired poxvirus such as MVA); wherein at least one epitope of the boosting composition is identical to an epitope of the priming composition. In one embodiment, the source of one or more hepatitis B epitopes in the priming composition is a DNA plasmid (e.g., pSG2.HBs). In another embodiment, the source of one or more hepatitis B epitopes in the priming composition is a viral vector, which is derived from a virus other than a non-replicating or replication-impaired poxvirus. In a further embodiment, the source of one or more hepatitis B epitopes is a non-replicating or replication impaired recombinant poxvirus; with the proviso that if the source of epitopes in the priming composition is a viral vector, the viral vector in the boosting composition is derived from a different virus. In a particular embodiment, the non-replicating or replication-impaired recombinant poxvirus is a Modified Vaccinia Virus Ankara (MVA) (e.g., MVA.HBs).

The present invention also encompasses a method of inducing an immune response against a (one or more) hepatitis B antigen (e.g., an antigen from the hepatitis B virus) in a mammal (e.g., human), which comprises administering to the mammal a priming composition (e.g., a DNA plasmid) comprising a source of one or more epitopes of the hepatitis B antigen. In a particular embodiment, the source of one or more hepatitis B epitopes in the priming composition is a DNA plasmid that is capable of expressing a hepatitis B antigen in a mammal (e.g., pSG2.HBs).

The present invention also includes an isolated plasmid comprising the nucleotide sequence of SEQ ID NO: 1. In addition, the invention provides an isolated recombinant replication-deficient poxvirus (e.g., MVA) comprising an insert, which comprises the nucleotide sequence of SEQ ID NO: 4 or SEQ ID NO: 5. The invention also encompasses compositions comprising an isolated plasmid comprising the nucleotide sequence of SEQ ID NO: 1, and compositions comprising an isolated recombinant replication-deficient poxvirus (e.g., MVA) comprising an insert, which comprises the nucleotide sequence of SEQ ID NO: 4 or SEQ ID NO: 5.

In one aspect the invention is a method of inducing an immune response against hepatitis B in a subject comprising the steps of

a) administering to the subject a priming composition comprising a DNA plasmid comprising a nucleotide sequence that is at least 90% homologous or identical to SEQ ID NO: 4 or SEQ ID NO: 5 followed by

b) administering to the subject a boosting composition comprising a recombinant MVA vector comprising a nucleotide sequence that is at least 90% homologous or identical to SEQ ID NO: 4 or SEQ ID NO: 5.

Also contemplated is a kit for inducing an immune response against hepatitis B in a subject comprising

a) a priming composition comprising a DNA plasmid comprising a nucleotide sequence that is at least 90% homologous or identical to SEQ ID NO: 4 or SEQ ID NO: 5 and

b) a boosting composition comprising a recombinant MVA vector comprising a nucleotide sequence that is at least 90% homologous or identical to SEQ ID NO: 4 or SEQ ID NO: 5.

Also contemplated is the use of

a) a priming composition comprising a DNA plasmid comprising a nucleotide sequence that is at least 90% homologous or identical to SEQ ID NO: 4 or SEQ ID NO: 5 and

b) a boosting composition comprising a recombinant MVA vector comprising a nucleotide sequence that is at least 90% homologous or identical to SEQ ID NO: 4 or SEQ ID NO: 5,

in the manufacture of a medicament for inducing an immune response against hepatitis B in a subject.

Preferably the DNA plasmid and/or the recombinant DNA vector comprise nucleotide sequence that is at least 95%, 98%, 99% or 100% homologous or identical to SEQ ID NO: 4 or SEQ ID NO: 5

Preferably the subject is a primate, more preferably a human.

In one embodiment the immune response is a memory T cell response. In a further embodiment the immune response is a CD8+ memory T cell response. In a further embodiment the immune response is a CD4+ memory T cell response.

### BRIEF DESCRIPTION OF THE DRAWINGS

FIG. 1 is a schematic of the construction of plasmid pTH.

FIG. 2 is a map of the pSG2 plasmid.

FIG. 3 is a schematic of the construction of plasmid pSG2.HBs.

FIG. 4 is a map of plasmid pSG2.HBs.

FIGS. 5A-5B show the nucleotide sequence (SEQ ID NO: 1) of plasmid pSG2.HBs.

FIGS. 6A-6B show the nucleotide sequence (SEQ ID NO: 2) of the HBV surface antigen (HBsAg) coding region in plasmid pSG2.HBs, and its predicted amino acid sequence (SEQ ID NO: 3).

FIG. 7 is an agarose gel showing restriction enzyme analysis of plasmid pSG2.HBs.

FIG. 8 is a map of the pSC 11 .HBs plasmid.

FIG. 9 is an agarose gel showing restriction enzyme analysis of plasmids pSC11 nd pSC11.HBs.

FIG. 10 is an agarose gel showing the PCR analysis of recombinant MVA.HBs using Hbs-specific primers (lanes 1-5) or MVA-specific primers (lanes 6-9).

FIG. 11 shows the expected (SEQ ID NO: 4) and actual (SEQ ID NO: 5) nucleotide sequence of the HBsAg gene insert in MVA.HBs, as determined by DNA sequence analysis.

FIG. 12 is a graph depicting levels of HBV DNA (“HBV”) and levels of alanine transferase activity (“ALT”) in serum samples from subject 102 of Group 1 during the course of treatment. Vertical arrows indicate when doses were administered.

FIG. 13 is a graph depicting levels of HBV DNA (“HBV”) and levels of alanine transferase activity (“ALT”) serum samples from subject 421 of Group A during the course of treatment. Vertical arrows indicate when doses were administered.

FIGS. 14A-D show the summed peptide responses after IVS Elispot (Example 8) from patients in Part 2 of the clinical trial study (Example 6). PMBC samples were taken at time points before, during and after therapy with heterologous PrimeBoost immunizations and/or lamivudine. Graphs show mean +/− standard deviation for each patient (A-C) and mean +/− s.e.m for each group (D).

FIG. 14A) 4 patients selected at random from Group A. Treated with immunizations with 2 mg DNA.HBs at weeks 0 and 3, immunizations with 5×10⁸ MVA.HBs at weeks 6 and 9, samples taken at week 0 (before therapy), 10 and 14 (after therapy). All patients show increased IVS ELISPOT responses after therapy, whereas only two of the four patients exhibited an ex vivo ELISPOT response (Table 12).

FIG. 14B) 3 patients selected at random from Group B. Treated with 100 mg/day lamivudine administered from week 0 to 14, immunized with 2 mg DNA.HBs at Weeks 4 and 7, immunizations with 5×10⁸ MVA.HBs at weeks 10 and 13, samples taken at week -2 (before lamivudine therapy), 4 (before commencing heterologous PrimeBoost immunizations), 14 and 18 (after therapy). Patient 519 showed increased ex vivo ELISPOT after PrimeBoost immunotherapy, patient 509 showed a moderate response after lamivudine and after the PrimeBoost immunotherapy, and patient 517 did not appear to respond to either lamivudine or the PrimeBoost immunotherapy.

FIG. 14C) 2 patients selected at random from Group C. Treated with 100 mg/day lamivudine administered from week 0 to 14, samples taken at week -2 (before lamivudine therapy), 4 (during lamivudine therapy) and 18 (after withdrawal of lamivudine). The patients each showed a mild transient response to lamivudine therapy.

FIG. 14D) Direct comparison of IVS ELISPOT responses for groups A-C. This is a superimposition of FIGS. 14A-C showing vaccinations and lamivudine administration.

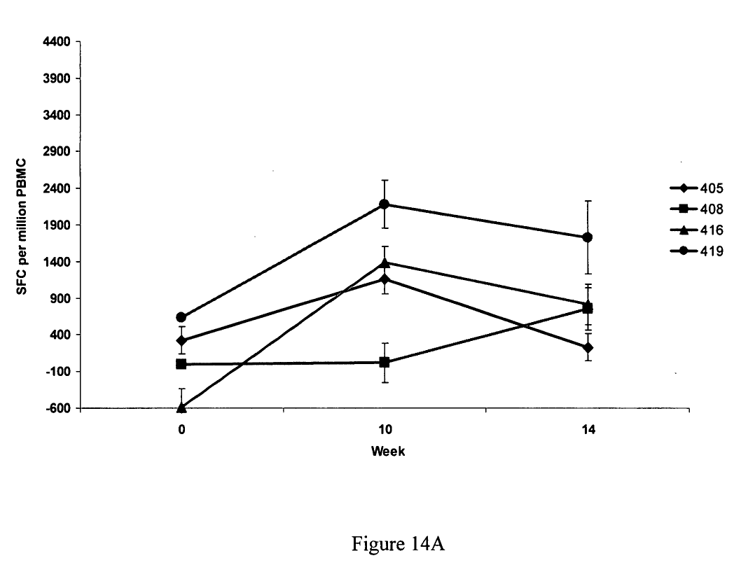

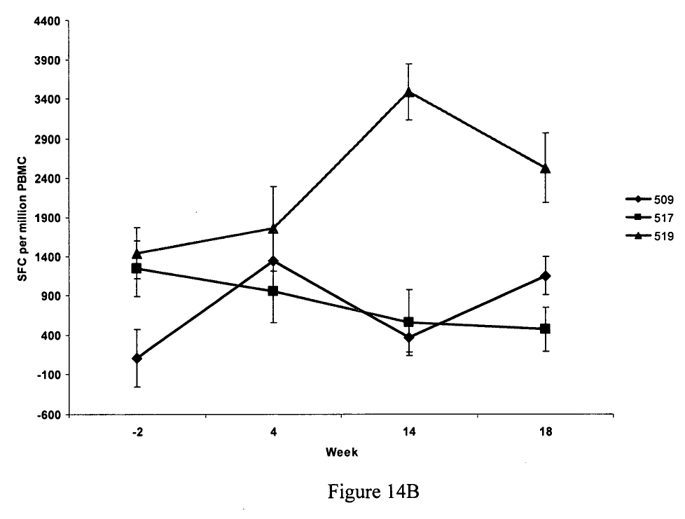

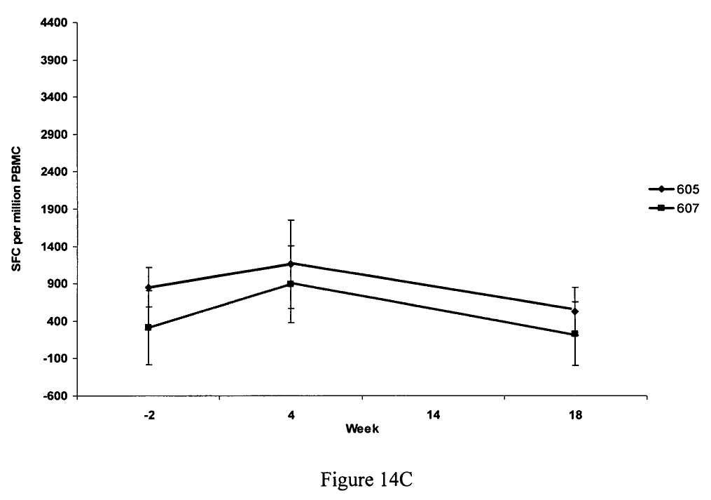

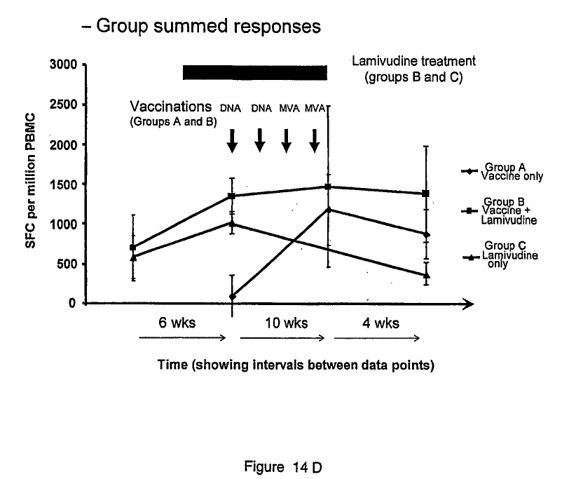

Mean ELISPOT values of the groups +/− s.e.m are shown. Bar shows the duration of 100 mg/day lamivudine treatment for Groups B and C. Arrows show dates of 2 mg DNA.HBs and 5×10⁸ pfu MVA.HBs vaccinations. Time axis is not to scale and not labeled due to the different start times used between Groups A and B-C (see FIGS. 14 A-C and Table 7). Intervals between the time points are consistent for the three groups and are shown (6 wks between first two data points, 10 wks between second and third data points, 4 wks between third and fourth data points). NB Group C is missing the third data point and Group A is missing the first data point.

### DETAILED DESCRIPTION OF THE INVENTION

The present invention is based, in part, on the discovery that a heterologous prime-boost regimen significantly potentiates immunological and clinical responses to a hepatitis B virus antigen in an individual.

Accordingly, the present invention is directed to a “prime-boost” administration regime, and involves the administration of at least two compositions:

(a) a first composition (priming composition) comprising a source of one or more epitopes of a hepatitis B target antigen; and

(b) a second composition (boosting composition) comprising a source of one or more epitopes of a hepatitis B target antigen, including at least one epitope which is the same as an epitope of the first composition.

The present invention also is based, in part, on the discovery that administration of a priming composition, which comprises a DNA plasmid that is capable of expressing one or more epitopes of a hepatitis B target antigen, significantly potentiates immunological and clinical responses to hepatitis B infection in an individual.

The methods of the present invention can be used to induce a “de novo” immune response against one or more hepatitis B antigens. Alternatively, the methods of the present invention can be used to boost a pre-existing immune response against one or more hepatitis B antigens.

As used herein, “mammal” and “mammalian” refer to any vertebrate animal, including monotreme, marsupials and placental, that suckle their young and either give birth to living young (eutherian or placental mammals) or are egg-laying (metatharian or nonplacental mammals). Examples of mammalian species include humans and primates (e.g., monkeys, chimpanzees), rodents (e.g., rats, mice, guinea pigs), ruminants (e.g., cows, pigs, horses), canines and felines. In a particular embodiment of the invention, the mammal is a human.

The methods described herein can induce, for example, an immune response that is a T cell immune response (e.g., CD8+ T cell, a CD4+ T cell) and/or a humoral (antibody) immune response. In one embodiment, the methods of the present invention induce a humoral (antibody) immune response. In another embodiment, the methods of the present invention induce a T cell immune response. In a particular embodiment, the immune response is a CD8+ T cell immune response. In another embodiment, the immune response is a CD4+ T cell immune response.

T cells fall into two major groups which are distinguishable by their expression of either the CD4 or CD8 co-receptor molecules. CD8-expressing T cells are also known as cytotoxic T cells by virtue of their capacity to kill infected cells or tumor cells. CD4-expressing T cells, on the other hand, have been implicated in mainly “helping” or “inducing” immune responses.

The nature of a T cell immune response can be characterised by virtue of the expression of cell surface markers on the cells. T cells in general can be detected by the presence of TCR, CD3, CD2, CD28, CD5 or CD7 (human only). CD4+ T cells and CD8+ T cells can be distinguished by their co-receptor expression (for example, by using anti-CD4 or anti-CD8 monoclonal antibodies, as is described in the Examples).

Since CD4+ T cells recognise antigens when presented by MHC class II molecules, and CD8+ recognise antigens when presented by MHC class I molecules, CD4+ and CD8+ T cells can also be distinguished on the basis of the antigen presenting cells with which they will react.

Within a particular target antigen, there may be one or more CD4+ T cell epitopes and one or more CD8+ T cell epitopes. If the particular epitope has already been characterised, this can be used to distinguish between the two subtypes of T cell, for example on the basis of specific stimulation of the T cell subset which recognises the particular epitope.

The induction of a T cell response will cause an increase in the number of the relevant T cell type. This may be detected by monitoring the number of cells, or a shift in the overall cell population to reflect an increasing proportion of CD4+ or CD8+ T cells. The number of cells of a particular type may be monitored directly (for example by staining using an anti-CD4+/CD8+ antibody, and then analysing by fluorescence activated cell scanning (FACScan)) or indirectly by monitoring the production of, for example a characteristic cytokine. CD4+ and CD8+ T cell responses are readily distinguished in ELISPOT assays by specific depletion of one or other T cell subset using appropriate antibodies. CD4+ and CD8+ T cell responses are also readily distinguished by FACS (fluorescence activated cell sorter) analysis.

The methods of the present invention comprise, administering to a mammal, an (one or more) epitope of a (one or more) hepatitis B target antigen. In a particular embodiment, the epitope is a T cell epitope (e.g., CD8+ T cell epitopes, CD4+ T cell epitopes). A T cell epitope is a short peptide derivable from a protein antigen. Antigen presenting cells can internalise antigen and process it into short fragments which are capable of binding MHC molecules. The specificity of peptide binding to the MHC depends on specific interactions between the peptide and the peptide-binding groove of the particular MHC molecule.

Peptides which bind to MHC class I molecules (and are recognised by CD8+ T cells) are usually between 6 and 12 amino acids, more usually between 8 and 10 amino acids in length. The amino-terminal amine group of the peptide makes contact with an invariant site at one end of the peptide groove, and the carboxylate group at the carboxy terminus binds to an invariant site at the other end of the groove. The peptide lies in an extended confirmation along the groove with further contacts between main-chain atoms and conserved amino acid side chains that line the groove. Variations in peptide length are accommodated by a kinking in the peptide backbone, often at proline or glycine residues.

Peptides which bind to MHC class II molecules are usually at least 10 amino acids, more usually at least 13 amino acids in length, and can be much longer. These peptides lie in an extended confirmation along the MHC II peptide-binding groove which is open at both ends. The peptide is held in place mainly by main-chain atom contacts with conserved residues that line the peptide-binding groove.

For a given antigen, CD4+ and CD8+ epitopes may be characterised by a number of methods known in the art. When MHC molecules are purified from cells, their bound peptides co-purify with them. The peptides can then by eluted from the MHC molecules by denaturing the complex in acid, releasing the bound peptide, which can be purified (for example by HPLC) and perhaps sequenced.

Peptide binding to many MHC class I and II molecules has been analyzed by elution of bound peptides and by X-ray crystallography. From the sequence of a target antigen, it is possible to predict, to a degree, where the Class I and Class II peptides may lie. This is particularly possible for MHC class I peptides, because peptides that bind to a given allelic variant of an MHC class I molecule have the same or very similar amino acid residues at two or three specific positions along the peptide sequence, known as anchor residues.

Also, it is possible to elucidate CD4+ and CD8+ epitopes using overlapping peptide libraries which span the length of the target antigen. By testing the capacity of such a library to stimulate CD4+ or CD8+ T cells, one can determine the which peptides are capable of acting as T cell epitopes.

The epitopes either present in, or encoded by the compositions, may be provided in a variety of different forms; such as a recombinant string of one or two or more epitopes, or in the context of the native target antigen, or a combination of both of these. Epitopes (e.g., CD4+ and CD8+ T cell epitopes) have been identified and can be found in the literature, for many different diseases. It is possible to design epitope strings to generate an immune response against any chosen antigen that contains such epitopes. Advantageously, the epitopes in a string of multiple epitopes are linked together without intervening sequences so that unnecessary nucleic acid and/or amino acid material is avoided. In addition to the epitopes from the target antigen, it may be preferable to include one or more other epitopes recognised by T helper cells or B cells, to augment the immune response generated by the epitope string. Particularly suitable T helper cell epitopes are ones which are active in individuals of different HLA types, for example T helper epitopes from tetanus (against which most individuals will already be primed).

The source of epitopes in the priming or boosting composition in the method according to the invention can be any suitable vehicle which can be used to deliver and/or express one or more epitopes of the target antigen in a mammal. For example, the source of epitopes in the priming or boosting composition in the method according to the invention can be a non-viral vector or a viral vector (e.g., a replicating viral vector, a non-replicating or replication-impaired viral vector).

In one embodiment, a heterologous prime-boost regimen is used to minimize cross reactivity between the source of epitopes used for the priming composition and the source of epitopes used for the boosting composition (see U.S. Pat. No. 6,663,871B1 and Published U.S. Application No. 2003/0138454, which are incorporated herein by reference). In this embodiment, the source of epitopes in the priming composition is different (heterologous) from the source of epitopes in the boosting composition. For example, in one embodiment, the source of epitopes in the priming composition is not a poxvirus vector, particularly when the boosting composition is a poxvirus vector, so that there is minimal cross-reactivity between the priming and boosting compositions. In a particular embodiment, the prime-boost regimen involves administering a priming composition that comprises the DNA plasmid, pSG2.HBs (described herein), followed by a boosting composition that comprises the recombinant virus, MVA.HBs (also described herein).

Alternative suitable viral vectors for use in the priming and boosting compositions according to the invention include a variety of different viruses, disabled so as to be non-replicating or replication-impaired. Such viruses include for example non-replicating adenoviruses such as E1 deletion mutants. Genetic disabling of viruses to produce non-replicating or replication-impaired vectors is well known.

Other suitable viral vectors for use in the priming and boosting compositions are vectors based on herpes virus and Venezuelan equine encephalitis virus (VEE). Suitable bacterial vectors for the priming composition include recombinant BCG and recombinant Salmonella and Salmonella transformed with plasmid DNA (Daxji A et al 1997 Cell 91: 765-775).

Alternative suitable non-viral vectors for use in the priming and boosting compositions include lipid-tailed peptides known as lipopeptides, peptides fused to carrier proteins such as KLH either as fusion proteins or by chemical linkage, whole antigens with adjuvant, and other similar systems.

In one embodiment of the invention, the source of epitopes in the priming and/or boosting compositions is a nucleic acid, which may be DNA or RNA, in particular a recombinant DNA plasmid. The DNA or RNA may be packaged, for example in a liposome, or it may be in free form. Nucleic acid molecules, including plasmids and vectors, according to the invention are normally provided in isolated, recombinant and/or purified form. Accordingly, hepatitis B sequences or other viral sequences are normally provided isolated from their natural environment, and may be free or substantially free of other hepatitis B nucleic acid sequences.

In one embodiment, the source of epitopes in the priming composition is a DNA plasmid (e.g., pSG2.HBs). In a particular embodiment, the source of epitopes in the priming composition is a DNA plasmid comprising the nucleotide sequence of SEQ. ID NO: 1. In another embodiment, the source of epitopes in the priming composition is a DNA plasmid comprising a nucleotide sequence that is at least 90%, 95%, 98% or 99% homologous to SEQ ID NO: 1.

In other embodiments the source of epitopes in the priming and/or boosting compositions comprise a nucleotide sequence that encodes the amino acid sequence of SEQ. ID NO. 3 or a fragment thereof. In another embodiment, the source of epitopes in the priming and/or boosting compositions comprise a nucleotide sequence that encodes an amino acid sequence that is at least 90%, 95%, 98% or 99% identical to the amino acid sequence of SEQ. ID NO. 3 or a fragment thereof. In a further embodiment, the source of epitopes in the priming and/or boosting compositions comprise a nucleotide sequence that encodes an amino acid sequence with up to 1, 3, 5, 10, or 20 amino acid additions, deletions or substitutions relative to the amino acid sequence of SEQ. ID NO: 3. For example, the source of epitopes may be a non-replicating or replication-impaired poxvirus vector such as a modified vaccinia virus Ankara (MVA), comprising a nucleotide sequence encoding SEQ ID NO: 3 or encoding an amino acid sequence at least 90, 95, 98 or 99% identical to SEQ ID NO: 3. Poxvirus vectors are especially preferred in boosting compositions of the invention, and are discussed in detail elsewhere herein.

In another embodiment of the invention, the source of epitopes in the priming and/or boosting compositions is a peptide, polypeptide, protein, polyprotein or particle comprising two or more epitopes, present in a recombinant string of epitopes or in a target antigen. Polyproteins include two or more proteins which may be the same, or different, linked together. The epitopes in or encoded by the priming or boosting composition are provided in a sequence which does not occur naturally as the expressed product of a gene in the parental organism from which the target antigen may be derived.

In another embodiment, the source of the epitopes in the boosting composition is a non-replicating or replication impaired recombinant poxvirus vector. In a particular embodiment, the source of epitopes in the boosting composition is a vaccinia virus vector such as MVA, NYVAC or a strain derived therefrom. Alternatives to vaccinia vectors include avipox vectors such as fowl pox or canarypox vectors. Particularly suitable as an avipox vector is a strain of canarypox known as ALVAC (commercially available as Kanapox), and strains derived therefrom. In a particular embodiment, the source of the epitopes in the boosting composition is a modified vaccinia virus Ankara (MVA) that comprises the nucleotide sequence of SEQ. ID NO: 4 or SEQ. ID NO: 5 (e.g., MVA.HBs). In another embodiment, the source of the epitopes in the boosting composition is a modified vaccinia virus Ankara (MVA) that comprises a nucleotide sequence that is at least 90%, 95%, 98% or 99% homologous to SEQ. ID NO: 4 or SEQ. ID NO: 5.

The term “non-replicating” or “replication-impaired” as used herein means not capable of replication to any significant extent in the majority of normal mammalian cells or normal human cells. Viruses which are non-replicating or replication-impaired may have become so naturally (i.e. they may be isolated as such from nature) or artificially e.g. by breeding in vitro or by genetic manipulation, for example deletion of a gene which is critical for replication. There will generally be one or a few cell types in which the viruses can be grown, such as CEF cells for MVA.

Replication of a virus is generally measured in two ways: 1) DNA synthesis and 2) viral titre. More precisely, the term “nonreplicating or replication-impaired” as used herein and as it applies to poxviruses means viruses which satisfy either or both of the following criteria:

1) exhibit a 1 log (10 fold) reduction in DNA synthesis compared to the Copenhagen strain of vaccinia virus in MRC-5 cells (a human cell line);

2) exhibit a 2 log reduction in viral titre in HELA cells (a human cell line) compared to the Copenhagen strain of vaccinia virus.

Examples of poxviruses which fall within this definition are MVA, NYVAC and avipox viruses, while a virus which falls outside the definition is the attenuated vaccinia strain M7.

Modified vaccinia virus Ankara (MVA) is a strain of vaccinia virus which does not replicate in most cell types, including normal human tissues. MVA was derived by serial passage >500 times in chick embryo fibroblasts (CEF) of material derived from a pox lesion on a horse in Ankara, Turkey (Mayr et al. Infection (1975) 33: 6-14.). It was shown to be replication-impaired yet able to induce protective immunity against veterinary poxvirus infections. MVA was used as a human vaccine in the final stages of the smallpox eradication campaign, being administered by intracutaneous, subcutaneous and intramuscular routes to >120,000 subjects in southern Germany. No significant side effects were recorded, despite the deliberate targeting of vaccination to high risk groups such as those with eczema (Mayr et al. Bakteriol B. (1978)167: 375-90).

The safety of MVA reflects the avirulence of the virus in animal models, including irradiated mice and following intracranial administration to neonatal mice. The non-replication of MVA has been correlated with the production of proliferative white plaques on chick chorioallantoic membrane, abortive infection of non-avian cells, and the presence of six genomic deletions totaling approximately 30 kb. The avirulence of MVA has been ascribed partially to deletions affecting host range genes K1 L and C7L, although limited viral replication still occurs on human TK-143 cells and African Green Monkey CV-1 cells. Restoration of the K1 L gene only partially restores MVA host range. The host range restriction appears to occur during viral particle maturation, with only immature virions being observed in human HeLa cells on electron microscopy (Sutter et al. 1992). The late block in viral replication does not prevent efficient expression of recombinant genes in MVA.

Poxviruses have evolved strategies for evasion of the host immune response that include the production of secreted proteins that function as soluble receptors for tumour necrosis factor, IL-I p, interferon (IFN)-α and IFN-γ, which normally have sequence similarity to the extracellular domain of cellular cytokine receptors (such as chemokine receptors).

These viral receptors generally inhibit or subvert an appropriate host immune response, and their presence is associated with increased pathogenicity. The I1-I p receptor is an exception: its presence diminishes the host febrile response and enhances host survival in the face of infection. MVA lacks functional cytokine receptors for interferon γ, interferon α, Tumour Necrosis Factor and CC chemokines, but it does possess the potentially beneficial IL-1 receptor. MVA is the only known strain of vaccinia to possess this cytokine receptor profile, which theoretically renders it safer and more immunogenic than other poxviruses. Another replication impaired and safe strain of vaccinia known as NYVAC is fully described in Tartaglia et al.(Virology 1992, 188: 217-232).

Poxvirus genomes can carry a large amount of heterologous genetic information. Other requirements for viral vectors for use in vaccines include good immunogenicity and safety. In one embodiment the poxvirus vector may be a fowlpox vector, or derivative thereof.

It will be evident that vaccinia virus strains derived from MVA, or independently developed strains having the features of MVA which make MVA particularly suitable for use in a vaccine, will also be suitable for use as an immunotherapeutic in the invention. In particular, MVA containing an inserted string of epitopes, or polyepitope gene, has been previously described in WO 98/56919.

The methods of the present invention can comprise administering one or more (a plurality) doses of the priming composition, followed by one or more doses of the first boosting composition to induce an immune response. In a particular embodiment, both the priming composition and boosting composition are administered in multiple doses.

The methods of the present invention also can comprise administering one or more (a plurality) doses of the priming composition to induce an immune response. In one embodiment, the priming composition is administered in multiple doses. In a particular embodiment, the priming compositions is administered twice.

The timing of the individual doses will depend on the individual. For example, the timing of the priming and boosting doses can be in the region of from about one week to three weeks, about 6 weeks to 9 weeks, about 9 weeks to 12 weeks, about 12 weeks to 15 weeks, about 15 to about 18 weeks and about 18 weeks to about 21 weeks apart. In particular embodiments, the timing of the priming and boosting doses can be about 2 weeks, 3 weeks, 4 weeks, 5 weeks, 6 weeks, 7 weeks, 8 weeks, 9 weeks, 10 week, 11 weeks, 12, weeks, 13 weeks, 14, weeks, 15 weeks, 16 weeks, 17 weeks, 18 weeks, 19 weeks, 20 weeks, 21 weeks, 22 weeks, 23 weeks, 24 weeks or 25 weeks apart.

The target antigen for use in the methods of the present invention can be any antigen that is characteristic of the target disease (e.g., hepatitis B). In one embodiment, the target antigen is derived from the hepatitis B virus (HBV). Suitable antigens from the hepatitis B virus include, but are not limited to, surface (e.g., -HBsAg) and/or core (e.g., HBcAg or HBeAg—soluble form) polymerase or x-protein, as well as any protein fragment thereof.

The target antigen may be an antigen which is recognized by the immune system after infection with the disease. Alternatively the antigen may be normally “invisible” to the immune system such that the method induces a non-physiological T cell response. This may be helpful in diseases where the immune response triggered by the disease is not effective (for example does not succeed in clearing the infection), as it may open up another line of attack.

The compositions described herein may be employed as therapeutic or prophylactic compositions (e.g., therapeutic compositions, immunotherapeutics, vaccines). Whether prophylactic or therapeutic immunisation is more appropriate will usually depend upon the nature of the disease.

The compositions of the present invention can exhibit a therapeutic effect when administered to a mammal, particularly a human. A therapeutic effect can be, among others, a decrease in HBV viral load or HBeAg in a subject's serum, an increase in IFN-gamma-secreting peptide specific T cells, elevation of the level of one or more liver enzymes, such as alanine transferase (ALT) and aspartate transferase (AST), and/or seroconversion of one or more HBV antigens. As used herein, seroconversion refers to the loss of one or more viral antigens from the serum of a subject, followed by the appearance of antibodies against the one or more viral antigens in the serum of the subject.

The methods of the present invention have been demonstrated using hepatitis B virus (HBV) antigens. In one embodiment, the method can be used to induce an immune response against multiple epitopes of HBV in a human. In a particular embodiment, the immune response is induced by an epitope of the HBV surface antigen (HBsAg). In a further embodiment, the immune response is induced against either the small form of the HBV surface antigen (small S antigen), the medium form of the HBV surface antigen (medium S antigen), or a combination thereof. In another embodiment, the immune response is induced by an epitope of the HBV surface antigen (HBsAg), in combination with other HBV-derived antigens, such as the core polymerase or x-protein.

Induction of an immune response against hepatitis B infection can be assessed using any technique that is known by those of skill in the art for determining whether an immune response has occurred. Suitable methods of detecting an immune response for the present invention include, among others, detecting a decrease in HBV viral load or HBeAg in a subject's serum, detection of IFN-gamma-secreting peptide specific T cells, and detection of elevated levels of one or more liver enzymes, such as alanine transferase (ALT) and aspartate transferase (AST). In one embodiment, the detection of IFN-gamma-secreting peptide specific T cells is accomplished using an ELISPOT assay.

In a particular embodiment, the induction of an immune response against hepatitis B is evidenced by seroconversion. In a further embodiment of the invention, seroconversion is detected during a period of less than 14 days after administration of the priming composition.

There is a clear need for the development of improved therapies for the treatment of hepatitis B. In order to test a prime-boost strategy for inducing anti-HBV CTL responses, recombinant DNA and vaccinia virus constructions are required.

To produce a DNA vector suitable for use in humans, plasmid pSG2 was constructed and validated by sequencing. This contains an enhancer/promoter/intron cassette for efficient expression of inserted antigens in mammalian cells, a polylinker cloning site, the bovine growth hormone transcription termination sequence, and sequences for propagation and selection in E. coli. The use of a kanamycin resistance marker avoids the risk of residual ampicillin-based contaminants in the manufactured product causing problems in sensitive individuals.

Plasmid pSG2.HBs was constructed by insertion of a 1,082 base pair (bp) fragment containing the pre-S2 and S genes of HBV into the polylinker cloning site of plasmid pSG2. Plasmid pSG2.HBs contains the CMV IE promoter with intron A, for driving expression of the HBV small S and medium S antigens in mammalian cells, followed by the bovine growth hormone transcription termination sequence. The plasmid also contains the kanamycin resistance gene and is capable of replication in E. coli but not in mammalian cells. The sequence of the poly-epitope gene was confirmed by sequencing, and both pSG2 and pSG2.HBs plasmids were characterised by restriction enzyme analysis. The complete sequence of plasmid pSG2.HBs was determined.

Plasmid pSG2.HBs contains genes encoding the small S and medium S antigen HBV epitopes under the control of an efficient promoter for expression in mammalian cells. The plasmid also carries sequences for propagation and selection in E. coli but is unable to replicate in mammalian cells. As shown herein, pSG2.HBs is a suitable DNA immunisation vector for use in humans.

Modified vaccinia virus Ankara (MVA) was selected as the vaccinia strain for development of a recombinant virus containing HBV epitopes. Recombinant MVA is considered to be a promising human vaccine candidate because of its safety profile and immunogenic properties.

Plasmid pSC11 (Chakrabarti et al 1985) contains the vaccinia late/early P7.5 promoter (Cochran et al 1985) to drive expression of the inserted antigen, and the vaccinia late promoter P11 driving expression of the lacZ marker gene. It also contains the left and right fragments of the vaccinia thymidine kinase (TK) gene flanking the region containing the lacZ gene and the inserted antigen so that these sequences can be inserted into the MVA genome by homologous recombination at the TK locus, thereby inactivating the TK gene.

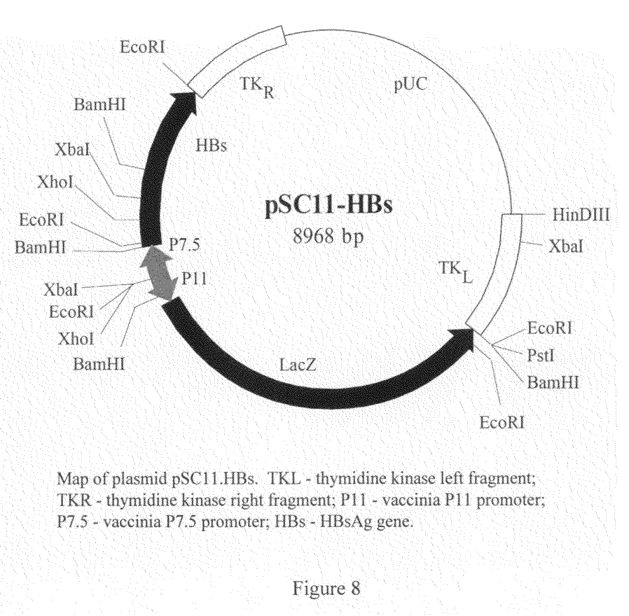

Plasmid pSC11.HBs was constructed by insertion of a HinDIII-NsiI fragment containing the pre-S2 and S genes of HBV into the polylinker region of plasmid pSC11 (FIG. 8). Plasmid pSC11.HBs therefore contains the vaccinia late/early P7.5 promoter driving expression of the inserted S antigen, and the vaccinia late promoter P11 driving expression of the lacZ marker gene, flanked by the left and right fragments of the vaccinia thymidine kinase (TK) gene.

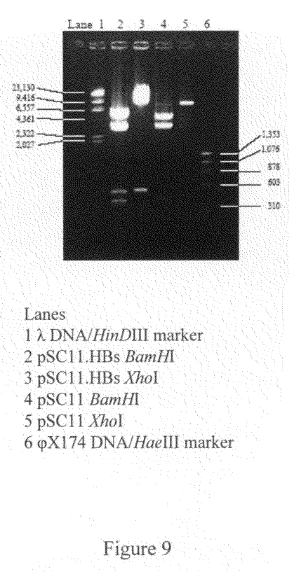

The DNA fragments generated following digestion of pSC11 and pSC11.HBs with restriction enzymes BamHI and XhoI is shown in FIG. 9.

Plasmid pCMVS2.S contains the pre-S2 and S sequences of HBV strain ayw. The plasmid contains a HinDIII site immediately 5′ to the pre-S2 gene and an NsiI site 3′ to the S gene. This fragment was isolated and treated with Kienow polymerase. This treatment filled in the overhang generated by cutting with HinDIII and removed the overhang generated by cutting with NsiI. The resulting 1,085 base pair (bp) blunt-ended fragment was inserted into the SmaI site of plasmid pSC11 to generate plasmid pSC11.HBs.

A novel immunotherapy comprising the DNA plasmid, pSG2.HBs, and the MVA viral vector, MVA.HBs, containing a source of epitopes from the HBV surface antigen, has been demonstrated. The data provided herein was designed to evaluate the safety and immnunogenicity of different doses and dosing regimens of a heterologous “PrimeBoost” immunisation schedule comprising pSG2.HBs “priming” followed by MVA.HBs “boosting” in subjects with chronic HBV infection.

This study evaluates the safety, immunogenicity and clinical response of increasing doses of DNA plasmid (pSG2.HBs) and MVA viral vector (MVA.HBs) containing a source of epitopes from the HBV surface antigen. The data indicate that pSG2.HBs and MVA.HBs are able to stimulate immunologically non-responsive patients as well as increase pre-existing immune responses.

The present invention is also directed to plasmids and recombinant viral vectors used in the methods described herein. In one embodiment, the invention is directed to an isolated plasmid comprising the nucleotide sequence of SEQ ID NO: 1. In another embodiment, the present invention is directed to an isolated recombinant replication-deficient poxvirus (e.g., MVA) comprising the nucleotide sequence of SEQ ID NO: 4 or SEQ ID NO: 5.

The priming and boosting compositions used in the method of the invention may conveniently be provided in the form of a “combined preparation” or kit. The priming and boosting compositions may be packaged together or individually for separate sale. The priming and boosting compositions may be used simultaneously, separately or sequentially for inducing an immune response against a target antigen.

The kit may comprise other components for mixing with one or both of the compositions before administration (such as diluents, carriers, adjuvants etc.—see below).

The kit may also comprise written instructions concerning the vaccination protocol, for example to administer the priming composition one or more times followed by the boosting composition one or more times.

In one embodiment, the kit comprises multiple (e.g. two) does of the priming composition and/or multiple (e.g. two) doses of the boosting composition, and instructions to administer the priming composition one or more times (e.g. twice) followed by the boosting composition one or more times (e.g. twice).

The present invention also relates to a product comprising the priming and boosting compositions as defined above. The product may be in the form of a pharmaceutical composition. A pharmaceutical composition may comprise a nucleic acid molecule or a virus according to the invention.

The pharmaceutical composition may also comprise, for example, a pharmaceutically acceptable carrier, diluent, excipient or adjuvant. The choice of pharmaceutical carrier, excipient or diluent can be selected with regard to the intended route of administration and standard pharmaceutical practice.

In particular, a composition comprising a DNA plasmid vector may comprise granulocyte macrophage-colony stimulating factor (GM-CSF), or a plasmid encoding it, to act as an adjuvant; beneficial effects are seen using GM-CSF in polypeptide form. Adjuvants such as QS21 or SBAS2 (Stoute J A et al. 1997 N Engl J Medicine 226: 86-91) may be used with proteins, peptides or nucleic acids to enhance the induction of T cell responses.

In the pharmaceutical compositions of the present invention, the composition may also be admixed with any suitable binder(s), lubricant(s), suspending agent(s), coating agent(s), or solubilising agent(s).

The pharmaceutical composition could be for veterinary (i.e. animal) usage or for human usage.

Nucleic acid molecules and viruses according to the invention may be used for treatment of the human or animal body by therapy, and especially for treating hepatitis B. Treatment includes preventative treatment such as vaccination.

In one aspect, the invention is use of a nucleic acid molecule or poxvirus of the invention in the manufacture of a medicament for treating hepatitis B. The medicament may be for inducing a de novo immune response, or for boosting a pre-existing immune response against hepatitis B in an individual. The medicament may be for administration in multiple doses, e.g. two doses. Medicaments comprising poxvirus are normally used as boosting compositions for administration following administration of a nucleic acid priming composition to an individual. Thus, in other aspects, the invention provides a priming composition and a boosting composition and a boosting composition in the manufacture of a medicament for sequential administration to an individual to treat hepatitis B. Suitable priming and boosting compositions are described in detail elsewhere herein.

In general, a therapeutically effective dose or amount of the compositions of the present invention is administered. The dosage for DNA compositions (e.g., DNA priming composition; DNA boosting composition) can be from about 0.5 mg to about 10 mg. In particular embodiments, the dosage for DNA compositions is from about 1 mg to about 4 mg. In a particular embodiment, the dosage for DNA compositions is about 2 mg. The dosage for vector (e.g., viral vector such as MVA) compositions (e.g., vector priming composition; vector boosting composition) can be from about 1×10⁷ pfu to about 1×10¹⁰ pfu. In particular embodiments, the dosage for vector compositions is from about 2×10⁷ to about 5×10⁹ pfu. In a particular embodiment, the dosage of the vector composition is from about 5×10⁷ pfu to about 1×10⁹.

The priming and boosting compositions of the present invention can be administered using any suitable route of administration. Tablets or capsules of the agents may be administered singly or two or more at a time, as appropriate. It is also possible to administer the compositions of the present invention in sustained release formulations.

Typically, the physician will determine the actual dosage which will be most suitable for an individual patient and it will vary with the age, weight and response of the particular patient. The above dosages are exemplary of the average case. There can, of course, be individual instances where higher or lower dosage ranges are merited, and such are within the scope of this invention.

Where appropriate, the pharmaceutical compositions can be administered by inhalation, by use of a gene gun, in the form of a suppository or pessary, topically in the form of a lotion, solution, cream, ointment or dusting powder, by use of a skin patch, orally in the form of tablets containing excipients such as starch or lactose, or in capsules or ovules either alone or in admixture with excipients, or in the form of elixirs, solutions or suspensions containing flavouring or coloring agents, or they can be injected parenterally, for example intracavernosally, intravenously, intramuscularly or subcutaneously. For parenteral administration, the compositions may be best used in the form of a sterile aqueous solution which may contain other substances, for example enough salts or monosaccharides to make the solution isotonic with blood. For buccal or sublingual administration the compositions may be administered in the form of tablets or lozenges which can be formulated in a conventional manner.

For some applications, preferably the compositions are administered orally in the form of tablets containing excipients such as starch or lactose, or in capsules or ovules either alone or in admixture with excipients, or in the form of elixirs, solutions or suspensions containing flavouring or colouring agents.

For parenteral administration, the compositions are best used in the form of a sterile aqueous solution which may contain other substances, for example enough salts or monosaccharides to make the solution isotonic with blood.

For buccal or sublingual administration the compositions may be administered in the form of tablets or lozenges which can be formulated in a conventional manner.

The physician will determine the actual dosage which will be most suitable for an individual patient and it will vary with the age, weight and response of the particular patient. It is to be noted that whilst the above-mentioned dosages are exemplary of the average case there can, of course, be individual instances where higher or lower dosage ranges are merited and such dose ranges are within the scope of this invention.

In some applications, generally, in humans, oral administration of the agents of the present invention is the preferred route, being the most convenient and can in some cases avoid disadvantages associated with other routes of administration—such as those associated with intracavernosal (i.c.) administration. In circumstances where the recipient suffers from a swallowing disorder or from impairment of drug absorption after oral administration, the drug may be administered parenterally, e.g. sublingually or buccally.

For veterinary use, the composition of the present invention is typically administered as a suitably acceptable formulation in accordance with normal veterinary practice and the veterinary surgeon will determine the dosing regimen and route of administration which will be most appropriate for a particular animal. However, as with human treatment, it may be possible to administer the composition alone for veterinary treatments.

### EXAMPLE 1

### Construction and Characterization of a Recombinant DNA Plasmid Expressing HBV Antigens

In order to test a prime-boost strategy for inducing an anti-HBV T-cell immune response, recombinant DNA and vaccinia virus constructs have been made containing a gene encoding the HBV surface antigen. The HBsAg gene of HBV has three potential initiation codons that divide the gene into pre-S1, pre-S2 and S regions. The small S antigen is encoded by the S region and is 226 amino acids in length. The medium S antigen is encoded by the S and pre-S2 regions and is 281 amino acids in length. This report describes the construction of a recombinant plasmid expressing the S and pre-S2 regions of HBV.

A DNA plasmid containing the S and pre-S2 regions of the HBsAg gene of

HBV was constructed and characterized.

### Materials and Methods

Materials and reagents

Buffers and reagents

### Buffers and Solutions:

Chemical reagents and buffers were purchased from Sigma.

### Enzymes and Molecular Biology Reagents:

Klenow polymerase (NEB)

T4 ligase (Promega M1801)

Restriction endonucleases (NEB)

DNA chromatography columns (Qiagen GmbH)

Agarose (Sigma A9539)

### Culture Reagents:

DH5a competent cells (Gibco 18258-012)

Bacterial growth media LB (Sigma L7275)

Ampicillin (Sigma A2804)

Kanamycin (Sigma K0879)

### DNA Reagents:

Oligonucleotides were purchased from R&D Systems Europe Ltd, 4-10 The Quadrant, Barton Lane, Abingdon, Oxon OX143YS.

Plasmid pRc/CMV was purchased from Invitrogen, PO Box 2312, 9704 CH Groningen, The Netherlands.

Plasmid pCMVS2.S was a gift from Dr H Davies (Loeb Medical Research Institute, Ottawa Civic Hospital, ON, Canada).

Plasmids pUC4K and pUC19 were purchased from Pharmacia, 100 Route, 206 North Peapack, N.J. 07977, USA.

Plasmid pEE14 contains the expression efficient enhancer/promoter/intron A cassette of the human cytomegalovirus (hCMV) strain AD169 (Whittle et al 1987).

Plasmid pSC11 was a gift from Dr E Cerundolo, Institute of Molecular Medicine, John Radcliffe Hospital, University of Oxford, Oxford OX3 9DS.

### Cells and Culture Medium:

The bacterial host strain used for DNA manipulation and propagation was Escherichia coli strain DH5α. Cells transformed with plasmid DNA containing the β-lactamase gene were propagated in LB liquid medium containing 50 μg/mL ampicillin or on plates containing the same medium plus 2% (w/v) agar. Cells transformed with plasmid DNA containing the kananycin resistance marker were propagated in LB liquid medium containing 25 μg/mL kanamycin or on plates containing the same medium plus 2% (w/v) agar.

### Experimental Methods

Unless stated otherwise, all DNA manipulations were carried out using standard molecular biology techniques as described in Current Protocols in Molecular Biology, Ed. F M Ausubel, John Wiley & Sons or according to the manufacturers' instructions.

Construction of Plasmid pSG2

Plasmid pSG2 was derived from plasmid pTH. To construct plasmid pTH, plasmid pRc/CMV was digested with BamHI and the fragments carrying the ColE1 origin of replication, β-lactamase (for ampicillin resistance), the hCMV promoter and the bovine growth hormone polyadenylation site were gel-purified. These fragments were re-ligated to create plasmid pCMVBam. This plasmid was then partially cut with BamHI, the single-cut DNA was gel-purified, the staggered ends filled in with Klenow polymerase and re-ligated. The resulting plasmid, containing a single BamHI site in the polylinker region was designated pCMV. The enhancer/promoter region of pCMV was then excised using MluI and HinDIII restriction endonucleases and a fragment from plasmid pEE14 containing the enhancer/intermediate early promoter/intron A region of hCMV was ligated between the sites to create plasmid pTH.

To construct plasmid pSG2, the amrpicillin resistance (Amp-r) marker in pTH was replaced with the kanamycin resistance marker (Kan-r) from the bacterial transposon Tn903 present in plasmid pUC4K. The Kan-r gene was excised from pUC4K on a BspHI fragment and ligated with pTH, also cut with BspHI which released the Amp-r gene. Subsequently, the plasmid underwent a spontaneous deletion of the sequence following the Kan-r gene. This deletion was found to be stable and did not affect function of the plasmid.

Construction of Plasmid pSG2.HBs

The source of the HBV sequence in plasmid pSG2.HBs was plasmid pCMVS2.S which contains the pre-S2 and S sequences of HBV strain ayw. The plasmid contains a HinDIII site immediately 5′ to the pre-S2 gene and an BamHI site 3′ to the S gene.

Insertion of the HBsAg sequence into pSG2 was carried out via the following steps. Firstly, pCMVS2.S was cut with HinDIII and BamHI to generate a fragment containing the 5′ end of the sequence. The 3′ end of the HBsAg sequence was isolated from pSC 1 .HBs as a BamHI-EcoRI fragment. These fragments were inserted into the polylinker region of plasmid pUC 19. The resulting plasmid was partially digested with HinDIII and EcoRI and the HBsAg fragment was isolated and inserted into plasmid pSG2, also partially cut with HinDIII and EcoRI. The overall insert size of the fragment inserted in pSG2.HBs is 1,082 bp (plus the 5′ HinDIII and 3′ EcoRI sites).

### Purification of Plasmid DNA

DNA plasmids were propagated in E. coli strain DH5α, purified using anion exchange chromatography columns (Qiagen) and resuspended in water. The concentration was calculated by spectrophotometric analysis at 260 nm and the DNA was then diluted in PBS.

Sequencing of Plasmid pSG2.HBs

The complete nucleotide sequence of plasmid pSG2.HBs was determined by Qiagen GmbH (Max-Volmer Str 4, 40724 Hilden, Germany).

Restriction Enzyme Analysis of Plasmid pSG2.HBs

Plasmid pSG2.HBs was digested with BarriHI and XhoI restriction enzymes (separate digests) and the resulting fragments were separated on an agarose gel at 100V for 30 minutes. Size markers used were φX174 DNA digested with HaeIII and XDNA digested with HinDIII. The expected size pattern of fragments (base pairs) generated by these digestions are:

| Restriction enzymes | pSG2.HBs |
|---|---|
| BamHI | 5413 |
| XhoI | 2927, 1164, 973, 324, 25 |

### Results

Construction of Plasmid pSG2

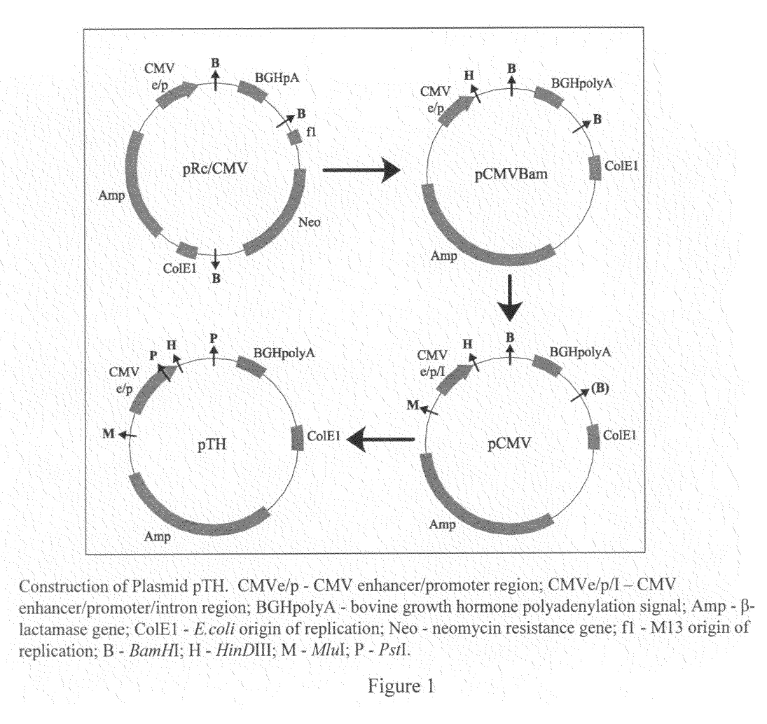

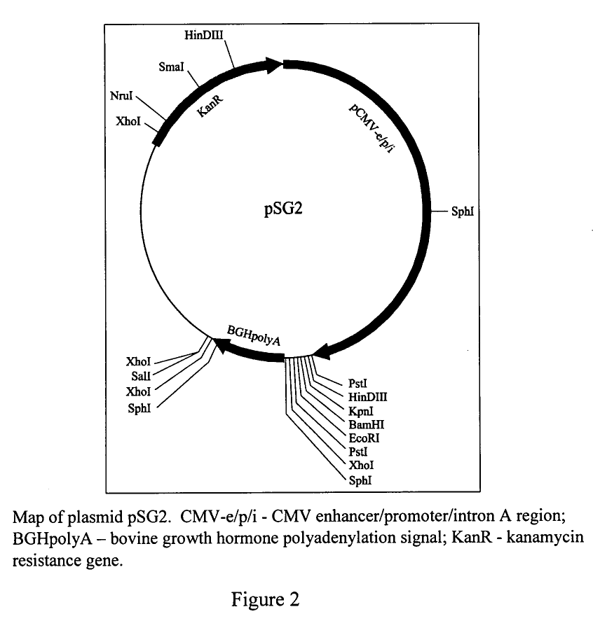

The construction of plasmid pTH is shown in FIG. 1. Plasmid pSG2 was derived from pTH by replacing the ampicillin resistance maker in pTH with a kanamycin resistance marker. A map of plasmid pSG2 is shown in FIG. 2. Construction of plasmid pSG2.HBs

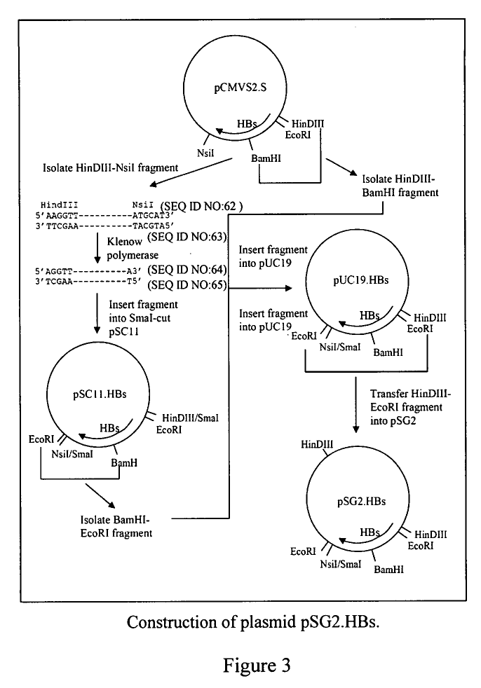

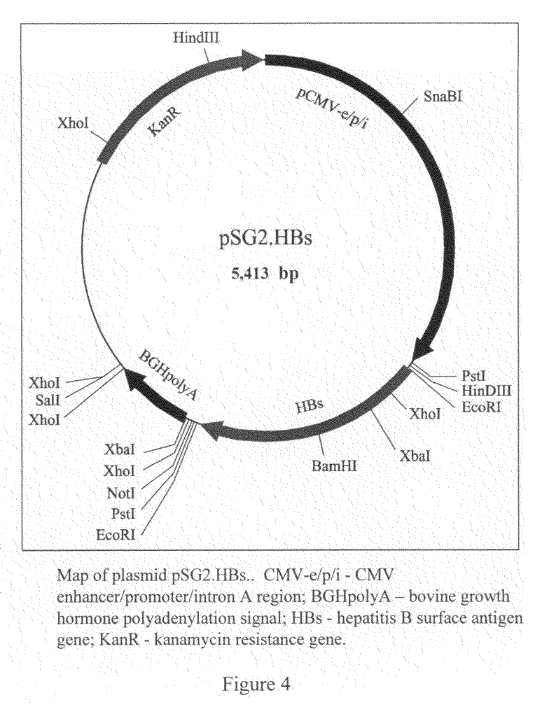

Plasmid pSG2.HBs was constructed by insertion of a 1,082 bp fragment containing the pre-S2 and S genes of HBV into the polylinker cloning site of plasmid pSG2. A summary of the cloning steps involved is shown in FIG. 3. Plasmid pSG2.HBs contains the CMV IE promoter with intron A for driving expression of the HBV pre-S2/S antigen in mammalian cells, followed by the bovine growth hormone transcription termination sequence. The plasmid also contains the kanamycin resistance gene and is capable of replication in E. coli but not in mammalian cells. A map of plasmid pSG2.HBs is shown in FIG. 4.

Characterisation of Plasmid pSG2.HBs
Complete Sequence of Plasmid pSG2.HBs

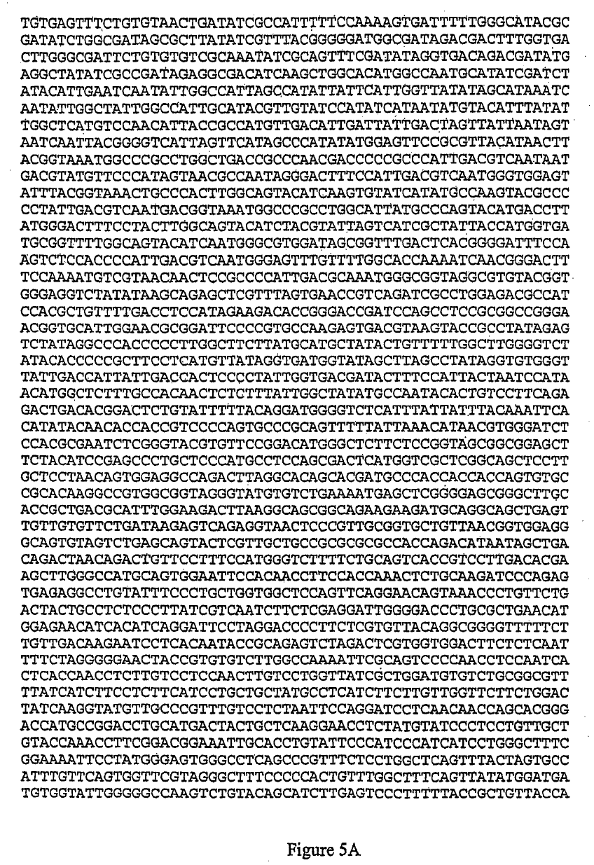

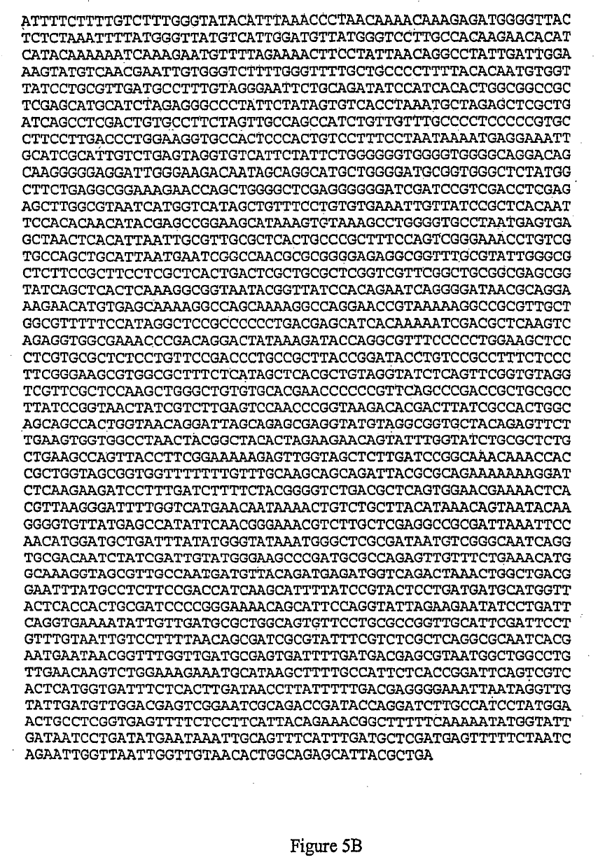

The complete sequence of plasmid pSG2.HBs is shown in FIGS. 5A-5B.

Sequence of the HBV S Antigen Gene in Plasmid pSG2.HBs

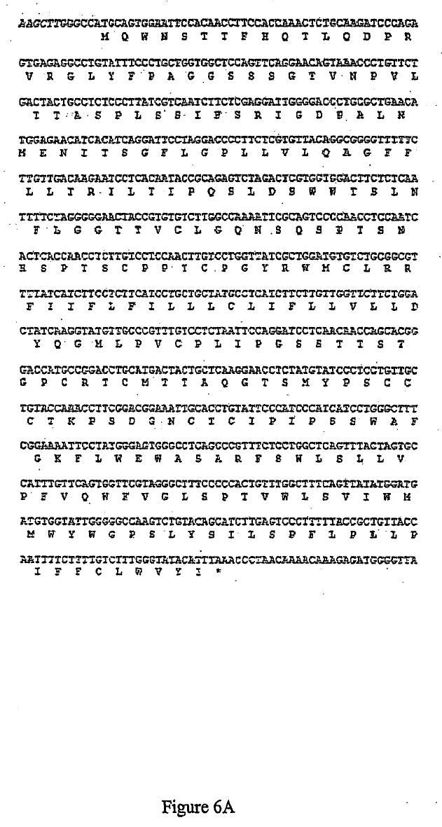

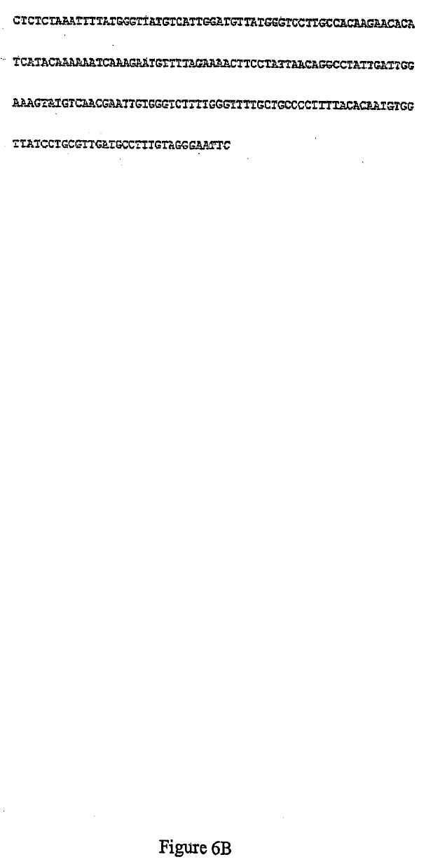

The sequence of the 1,082 bp DNA insert in plasmid pSG2.HBs is shown in FIGS. 6A-6B. The insert contains an 843 bp open reading frame (encoding the pre-S2 and S regions of the HBsAg gene) followed by a translation stop codon and a 3′ untranslated region.

Restriction Enzyme Analysis of Plasmid pSG2.HBs

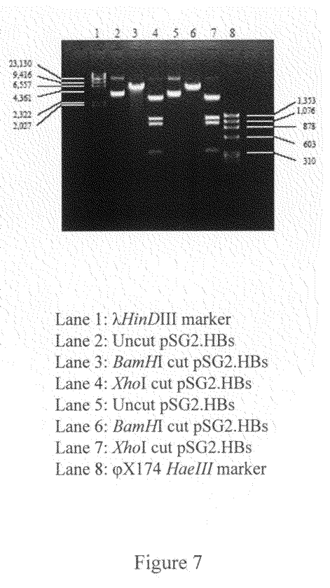

The DNA fragments generated following digestion of plasmid pSG2.HBs with restriction enzymes BamHI and XhoI is shown in FIG. 7.

### Discussion

Plasmid pSG2.HBs was generated by insertion of a gene fragment containing the pre-S2 and S sequences of HBV strain ayw into the polylinker cloning region of pSG2. The complete sequence of plasmid pSG2.HBs was determined and the plasmid is 5,413 bp in size.

Plasmid pSG2.HBs was also characterised by restriction enzyme analysis. The pattern of fragments generated, and their sizes, were consistent with the predicted pattern based on the sequence of the plasmid.

Plasmid pSG2.HBs contains the pre-S2 and S regions of the HBsAg gene of HBV strain ayw under control of an efficient promoter for expression in mammalian cells. The plasmid also carries sequences for propagation and selection in E. coli but is unable to replicate in mammalian cells. It is, therefore, a suitable DNA immunisation vector for use in humans.

### EXAMPLE 2

### Construction and Characterisation of Recombinant MVA Expressing HBV Antigens

A variety of attenuated recombinant viral vectors have been developed as antigen delivery systems. However, not all attenuated viruses are replication-incompetent in mammalian hosts and the use of attenuated but replication-competent viruses can lead to side effects, particularly in immunocompromised individuals.

Modified vaccinia virus Ankara (MVA) is a strain of vaccinia virus that does not replicate in most cell types, including normal human tissues (Mayr et al 1978). MVA was derived by multiple passages of a vaccinia virus from a horse pox lesion and was administered to 120,000 people in the last stages of the smallpox eradication program in Germany. The genome of MVA has been fully sequenced and the virus has six genomic deletions totalling 30 kb. The avirulence of MVA has been ascribed in part to deletions of host range genes and it also lacks several genes coding for immunomodulatory proteins. Since infection with replication-impaired viruses is abortive and therefore delivers a lower dose of antigen in vivo, it has been speculated that these viruses would be less immunogenic than their replication-competent parental strains. However, in studies comparing replication-impaired vaccinia viruses with a replication-competent virus, only boosting DNA-primed animals with replication-impaired poxviruses induced high levels of protection against malaria (Schneider et al 1998). Recombinant MVA is therefore considered to be a promising human vaccine candidate because of its safety profile and immunogenic properties.

In order to test a prime-boost strategy for inducing anti-HBV CTL responses, recombinant DNA and vaccinia virus constructions have been made containing a gene encoding the HBV surface antigen (HBsAg). The HBsAg gene of HBV has three potential initiation codons that divide the gene into pre-S1, pre-S2 and S regions. The small S antigen is encoded by the S region and is 226 amino acids in length. The medium S antigen is encoded by the S and pre-S2 regions and is 281 amino acids in length. This report describes the construction of a recombinant MVA expressing the S and pre-S2 regions of HBV.

A recombinant MVA containing the S and pre-S2 regions of the HBsAg gene of HBV was constructed and characterized.

### Materials and Methods

Materials and reagents

Buffers and reagents

### Buffers and Chemicals:

Chemical reagents and buffers were purchased from Sigma

### Enzymes and Molecular Biology Reagents:

T4 ligase (Promega M1801)

Restriction endonucleases (NEB)

DNA chromatography columns and buffers (Qiagen GmbH)

Agarose (Sigma A9539)

Ethidium bromide (Sigma E-151)

2×Reddy master mix 2.5 mM MgCl₂ (AB Gene AB-0619/LD/DC)

### Bacterial Culture Reagents:

Bacterial growth medium LB (Sigma L7275)

Ampicillin (Sigma A2804)

Kanamycin (Sigma K0879)

DH5a competent cells (Gibco 18258-012)

### Tissue Culture Reagents:

Fetal calf serum (FCS) (Sigma)

Superfect (Qiagen)

MEM (Sigma)

Penicillin (100 units) (Sigma)

Streptomycin (100 μg/mL) (Sigma)

Tissue culture plates (Falcon)

X-gal (Promega)

Formaldehyde (37%) (Sigma)

Carboxymethyl cellulose (CMC) (BDH 276494N)

CMC overlay: Prepare 3% CMC in water and autoclave. Mix 1:1 with 2×MEM containing 4% FCS and 2×penicillin/streptomycin.

### Immunoassay Reagents:

96-well nitrocellulose plates (Milliscreen MAHA, Millipore, UK)

24-well plates (Coming Costar)

Bovine serum albumin (BSA) (Sigma)

Fast DAB kit (Sigma D-0426)

Anti-mouse IgG peroxidase conjugate (Sigma Immunochemicals A-2554)

Anti-MVA antibody Mouse serum from BALB/c mice immunised twice with 1×10⁶ plaque-forming units (pfu) of MVA

### DNA and Viral Reagents

Oligonucleotides were purchased from R&D Systems Europe Ltd, 4-10 The Quadrant, Barton Lane, Abingdon, Oxon OX14 3YS or from MWG Biotech AG, Anzinger Strasse 7, D-85560 Ebersberg, Germany.

Plasmid pCMVS2.S was a gift from Dr H Davies (Loeb Medical Research Institute, Ottawa Civic Hospital, ON, Canada).

Plasmid pSC11 was a gift from Dr E Cerundolo, Institute of Molecular Medicine, John Radcliffe Hospital, University of Oxford, Oxford OX3 9DS.

Non-recombinant MVA was obtained from Anton Mayr, University of Munich Germany.

### Cells and Culture Medium

The bacterial host strain used for DNA manipulation and propagation was Escherichia coli strain DH5α. Cells transformed with plasmid DNA containing the β-lactamase gene were propagated in LB liquid medium containing 50 μg/mL ampicillin or on plates containing the same medium plus 2% (w/v) agar. Cells transformed with plasmid DNA containing the kanamycin resistance marker were propagated in LB liquid medium containing 25 μg/mL kanamycin or on plates containing the same medium plus 2% (w/v) agar.

Recombinant and non-recombinant vaccinia viruses were routinely propagated in primary chicken embryo fibroblasts (CEFS) grown in minimal essential medium (MEM) supplemented with 10% (v/v) foetal calf serum (FC S). For growth, CEFs were cultivated in MEM with 10% (v/v) FCS and incubated at 37₁C. For maintenance, CEFs were incubated in MEM with 2% (v/v) FCS and incubated at 30₁C. MVA is unable to infect cells at a FCS concentration of 10% (v/v). For infection, CEFs are therefore grown in MEM with 10% (v/v) FCS, rinsed in phosphate-buffered saline (PBS) and virus added in MEM containing 2% (v/v) FCS.

### Experimental Methods

Unless stated otherwise, all DNA manipulations were carried out using standard molecular biology techniques as described in Current Protocols in Molecular Biology, Ed. F. M. Ausubel, John Wiley & Sons or according to the manufacturers' instructions.

Construction of Pasmid pSC11.HBs

Plasmid pSC11 (Chakrabarti et al 1985) contains the vaccinia late/early P7.5 promoter (Cochran et al 1985) to drive expression of the inserted antigen, and the vaccinia late promoter P11 driving expression of the lacZ marker gene. It also contains the left and right fragments of the vaccinia thymidine kinase (TK) gene flanking the region containing the lacZ gene and the inserted antigen so that these sequences can be inserted into the MVA genome by homologous recombination at the TK locus, thereby inactivating the TK gene.

Plasmid pCMVS2.S contains the pre-S2 and S sequences of HBV strain ayw. The plasmid contains a HinDIII site immediately 5′ to the pre-S2 gene and an NsiI site 3′ to the S gene. This fragment was isolated and treated with Klenow polymerase. This treatment filled in the overhang generated by cutting with HinDIII and removed the overhang generated by cutting with NsiI. The resulting 1,085 base pair (bp) blunt-ended fragment was inserted into the SmaI site of plasmid pSC11 to generate plasmid pSC11.HBs (FIG. 8).

### Purification of Plasmid DNA

DNA plasmids were propagated in E. coli strain DH5α, purified using anion exchange chromatography columns (Qiagen) and resuspended in water. The concentration was calculated by spectrophotometric analysis at 260 nm and the DNA was then diluted in PBS.

Restriction Enzyme Analysis of Plasmids pSC11 and pSC11.HBs

Plasmids pSC11 and pSC11.HBs were digested with BamHI and XhoI (separate digests) and the resulting fragments were separated on an agarose gel at 100V for 40 minutes. Size markers used were φX174 DNA/HaeIII and k DNA/HinDIII. The expected size pattern of fragments (base pairs) generated by these digestions were:

| Restriction enzymes | pSC11 | pSC11.HBs |
|---|---|---|
| BamHI | 4426, 3071, 386 | 4996, 3071, 515, 386 |
| XhoI | 7883 | 8434, 534 |

### Selection of MVA.HBs

Recombinant viruses were produced by infecting primary CEFs with MVA, then transfecting the same cells with the appropriate shuttle vector. CEF cultures (90% confluent) were infected with 1-2 pfu/cell wild type MVA in 1 mL MEM with 2% (v/v) FCS for 120 minutes in a standard tissue culture incubator (37° C., 5% CO₂). After infection, the cells were transfected with pSC11.HBs using Superfect. Following a two hour incubation the cells were incubated for 2 days in MEM with 2% (v/v) FCS to allow recombination and viral replication to occur.

Wild type and recombinant viruses were released by repeated freeze/thawing of the cells (3 times in a dry ice/isopropanol bath). The virus mixture was diluted (undiluted, 10-1, 10-2 10-3) in MEM with 2% (v/v) FCS and plated out on fresh CEF monolayers. The monolayers were overlaid with 2 mL of agarose (2% (w/v) low melting point agarose mixed 1:1 with 2×MEM containing 4% (v/v) FCS and 2×penicillin/streptomycin). Following 48 hours incubation a further overlay of agarose containing X-gal (0.25 μg/mL) was added. After overnight incubation blue-stained areas containing recombinant virus expressing LacZ could be identified. These were isolated by picking areas containing agarose and the underlying infected cells with Gilson P1000 pipette tips. Virus was released by freeze thawing (3 times), diluted, sonicated and re-plated as described. This procedure was repeated five times. Preparation of MVA.HBs virus stock After 5 rounds of plaque purification, two 75cm² tissue culture flasks (T-75) of CEFs were infected with the plaque-purified material. A cytopathic effect (CPE) was observed and the virus was harvested 3 days after infection. The virus was amplified further in 5 T-150 flasks, harvested and titrated. Harvested virus was stored at −70° C.

### Bulk Virus Preparation

Recombinant MVA.HBs was grown on primary CEFs (ten T-150 flasks with CEFs almost confluent). Maximum CPE was visible after 3 days and the cells were harvested. Virus content was determined by titration. The material was diluted to 1×10⁷ pfu/mL in 10 mM Tris (pH 9.0).

To test the sterility of the bulk virus preparation, 200 μL of the diluted stock was inoculated into LB medium and incubated overnight.

### Titration of the Virus Stock by X-Gal Staining

CEFs were plated into 24-well plates (4×10⁵ cells per well) and incubated overnight to obtain the required confluency. The virus stock was diluted (10-2, 10-3, 10-4, 10-5, 10-6, 10-7 and 10-8) in MEM with 2% (v/v) FCS and 100 μL aliquots were distributed into 4 wells. Following incubation for 1 hour at 37° C. each well was overlaid with approximately 0.4 mL CMC overlay. Plates were incubated for 48 hours. Wells were filled with 1% (v/v) formaldehyde and the cells were fixed for 5 minutes. All liquid was then removed and the wells were washed with PBS (1 mL/well).

For one plate, the following staining solution was prepared (in this order) and

0.4 mL added to each well:

9.73 mL H₂O

0.1 mL 0.5 M K₃Fe(CN)₆

0.1 mL 0.5 M K4Fe(CN)₆.3H₂O

0.02 mL 1 M MgCl₂

0.05 mL 50 mg/mL X-gal in dimethylformamide

Blue spots developed after 1 hour at room temperature. The spots were counted in wells where they were well separated, taking the average of the 4 wells prepared from the same dilution. The titre was calculated as follows:

Average number of spots×dilution factor×10=pfu/mL

### PCR Analysis of MVA.HBs

The absence of non-recombinant MVA in the virus stock was assessed by PCR, using two sets of primers that would distinguish between recombinant and non-recombinant virus. The PCR reactions were carried out using commercially available reagents and the primers described herein. The products of the PCR reactions were analysed by agarose gel electrophoresis followed by staining with ethidium bromide. The bands generated were compared with appropriate size markers (HaeIII-digested φX174 DNA). The predicted size of the PCR fragment from non-recombinant MVA is 419 bp and the predicted size of the PCR fragment from MVA.HBs is 440 bp.

### Sequencing of Inserted DNA in MVA.HBs

The HBs sequence inserted at the TK locus of MVA was isolated by PCR using. The sequence of the PCR fragment was determined by MWG Biotech AG, Germany. The oligonucleotide primers used are listed below.

PCR Primers
Oligonucleotide primers used for PCR analysis of
MVA.HBs
HBSU2:

(SEQ ID NO: 6)

5′-TCCTGCGTTGATGCCTTTGTA-3′
TKU:

(SEQ ID NO: 7)

5′-CAATTACAGATTTCTCCGTGATAGGT-3′
TKL:

(SEQ ID NO: 8)

5′-TCATTTGCACTTTCTGGTTCGTA-3′
Oligonucleotide primers used for isolating the HBs
antigen gene from MVA.HBs
FSEQ:

(SEQ ID NO: 9)

5′-GTAAAACGACGGCCAGTTTGCACGGTAAGGAAGTAGAATCAT-3′
ERSEQ:

(SEQ ID NO: 10)

5′-AACAGCTATGACCATGTTCCTTGGTTTGCCATACGCTC-3′
Oligonucleotide primers used for sequencing of the
HBs antigen gene in MVA.HBs
M13/pUC universal forward primer:

(SEQ ID NO: 11)

5′-GTAAAACGACGGCCAGT-3′
M13/pUC universal reverse primer:

(SEQ ID NO: 12)

5′-CACACAGGAAACAGCTATGACCAT-3′
III656a1:

(SEQ ID NO: 13)

5′-GGTCCTAGGAATCCTGATG-3′
III656a2:

(SEQ ID NO: 14)

5′-GGATGTTATGGGTCCTTGC-3′

### Results

Construction and Characterisation of Plasmid pSC11 .HBs

Plasmid pSC11.HBs was constructed by insertion of a HinDIII-NsiI fragment containing the pre-S2 and S genes of HBV into the polylinker region of plasmid pSC11 (FIG. 8). Plasmid pSC11.HBs therefore contains the vaccinia late/early P7.5 promoter driving expression of the inserted S antigen, and the vaccinia late promoter P11 driving expression of the lacZ marker gene, flanked by the left and right fragments of the vaccinia thymidine kinase (TK) gene.

The DNA fragments generated following digestion of pSC11 and pSC11.HBs with restriction enzymes BamHI and XhoI is shown in FIG. 9.

### Construction and Characterization of MVA.HBs

### Selection and Purification of Recombinant MVA.HBs

A recombinant MVA.HBs virus was isolated by 5 rounds of plaque purification following infection/transfection of CEF cells with wild type MVA and pSC11.HBs. A stock of virus was purified by sucrose density centrifugation. However, further analysis of this stock indicated that it contained non-recombinant MVA in addition to recombinant MVA.HBs. A second round of plaque purification was therefore initiated using virus purified by Impfstoffwerk Dessau-Tomau GmbH (IDT, Germany) as the starting material. Following a further 5 rounds of plaque purification, a single plaque was used to infect two T-75 flasks of CEFs. Cells were harvested and the virus was amplified further by using this material to infect five T-150 flasks. Cells were harvested and the virus titre was determined to be 1.25×10⁸ pfu/mL (total 2.5 mL).

### Bulk Virus Preparation

Ten T-150 flasks were infected with the plaque-purified virus stock. Cells were harvested and the virus titre was determined to be 2.2×10⁹ pfu/mL (total 5 mL). This material was diluted to 1×10⁷ pfu/mL and its sterility confirmed by inoculation of LB medium.

### PCR Analysis

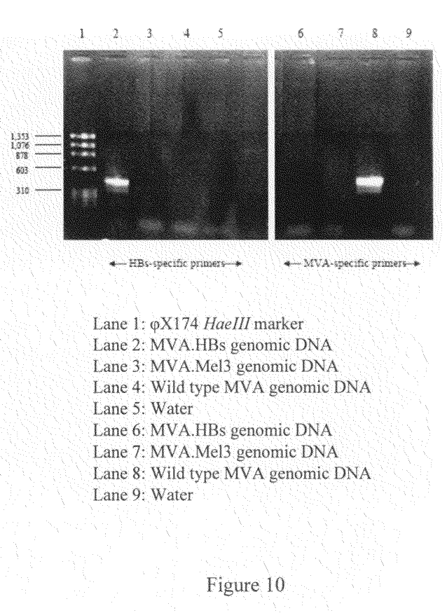

The purity of the recombinant MVA.HBs was assessed using PCR analysis. The sterile bulk virus preparation (1×10⁷ pfu/mL) was supplied to IDT where it was used to further amplify the virus. This material was then returned to Oxxon Pharmaccies for PCR analysis. Viral genomic DNA was extracted and the absence of nonrecombinant virus was confirmed (FIG. 10). A band was detected in the reaction mixture containing MVA.HBs genomic DNA plus the HBs-specific primers (FIG. 10, lane 2). No bands were detected in the reaction mixtures containing either genomic DNA from an irrelevant recombinant MVA (MVA.Mel3) or non-recombinant genomic DNA (FIG. 10, lanes 3 and 4). A band was detected in the reaction mixture containing nonrecombinant MVA genomic DNA plus the MVA-specific primers (FIG. 10, lane 8). No bands were detected in the reaction mixtures containing either MVA.HBs genomic DNA or genomic DNA from an irrelevant recombinant MVA (MVA.Mel3) (FIG. 10, lanes 6 and 7).

### Sequencing of the Inserted HBsAg Gene

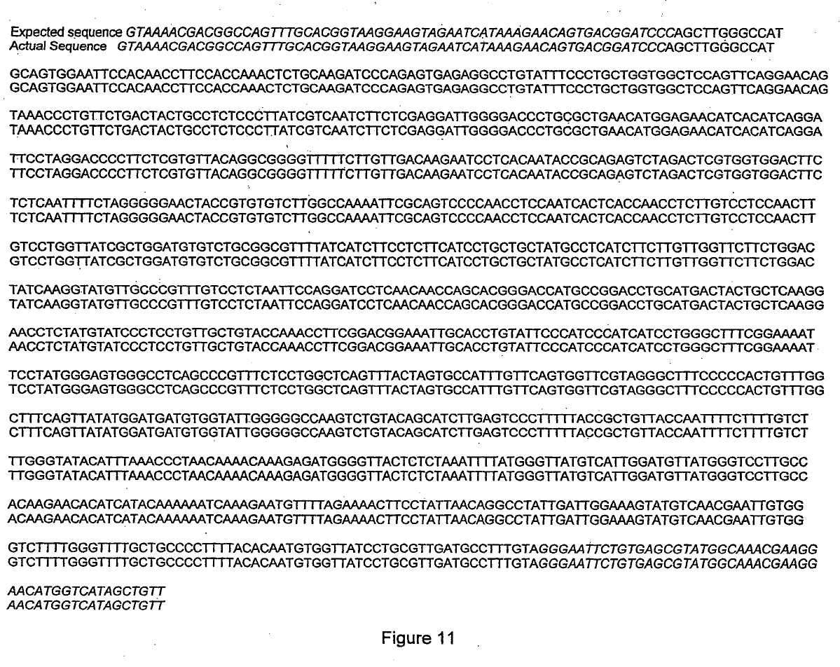

The inserted gene in MVA.HBs was determined and was consistent with the predicted sequence (FIG. 11).

### Discussion

The transfer vector pSC11.HBs was generated by insertion of a gene fragment containing the pre-S2 and S sequences of HBV strain ayw between the left and right fragments of the vaccinia thymidine kinase (TK) gene present in plasmid pSC11. Plasmid pSC11 also contains the lacZ gene between the flanking TK regions. Plasmid pSC11.HBs was characterised by restriction enzyme analysis. The pattern of fragments generated, and their sizes, were consistent with the predicted pattern based on the sequence of the plasmid.

Transfecting CEF cells infected with wild-type MVA with pSC11.HBs resulted in homologous recombination across the TK sequences. Recombinant virus was identified by the presence of blue plaques due to the expression of LacZ. Recombinant virus was plaque-purified 5 times and a stock of MVA.HBs was prepared. Analysis of this stock indicated that it also contained non-recombinant MVA and a further 5 rounds of plaque purification were therefore carried out. A virus stock was produced and characterised by titration using X-gal staining (titre 1.25×10⁸ pfu/mL). A bulk virus preparation was also made. The purity of the recombinant virus was confirmed by PCR analysis.

The MVA.HBs was also characterised by sequencing the HBV gene. The sequence obtained was consistent with the predicted sequence.

Recombinant MVA.HBs contains the pre-S2 and S regions of HBV strain ayw under control of the vaccinia P7.5 promoter. It also contains the bacterial lacZ marker gene under the control of the vaccinia P11 promoter. MVA is a strongly attenuated vaccinia virus strain and the recombinant MVA.HBs virus should therefore be a suitable immunisation vector for use in humans.

### EXAMPLE 3

### Induction of Specific CD8+ T cell Responses Against the Hepatitis B Virus Surface Antigen in BALB/c Mice

To evaluate the ability of the pSG2.HBs and MVA.HBs immunotherapeutic to induce specific CD8+ T cell responses against the hepatitis B virus surface antigen (HBsAg), BALB/c mice were immunized intramuscularly with pSG2.HBs (50 μg) or intradermally with MVA.HBs (5×10⁶ pfu). In BALB/c mice, the peptide IPQSLDSWWTSL (SEQ ID NO: 15) is recognized by CD8+ T cells as the immunodominant epitope. Using this peptide, CD8+ T cells were assayed using two different methods:

a cytotoxicity assay involving in vitro restimulation with peptide followed by a standard ⁵¹Cr release lysis assay;

an ELISPOT assay to determine the number of IFN-gamma secreting peptide-specific CD8+ T cells (spot forming cells=SFC).

### Materials and Methods

### Cytotoxicity Assay

Single cell suspensions of splenocytes were prepared as follows. Individual spleens were macerated and the suspension filtered through a cell strainer. The cells were pelleted and red blood cells were lysed using a hypotonic buffer. The splenocytes were resuspended in buffer and restimulated for 5-8 days with an HBsAg peptide (IPQSLDSWWTSL).

The peptide-specific cytotoxic activity was determined using a standard ⁵¹Cr release assay, in which peptide-restimulated splenocytes are tested for their ability to lyse peptide-pulsed syngeneic target cells (P815 cells). Cytotoxicity was tested on P815 cells pulsed with either the relevant HbsAg peptide (IPQSLDSWWTSL) or with an irrelevant peptide. Target cells were incubated with Na⁵¹Cr and peptides for 1 hour at 37° C. After washing, the target cells were coincubated (four hours) with effector cells at decreasing ratios of effector to target (E:T) cells. The E:T ratios tested were:—100: 1, 33:1, 11:1, 3:1 and 1:1. For determination of minimum and maximum ⁵¹Cr-release, target cells were incubated with medium alone or with 0.3% Triton-X100, a detergent that lyses the cell membranes. Release of ⁵¹Cr was measured in a beta counter. Percent specific lysis was determined according to the following formula:

$$\% specific lysis = \frac{( sample release - medium release )}{( maximum release - medium release )}$$

### ELISPOT Assay

Microtitre plates were coated with rat anti-mouse IFN antibody and then splenocytes isolated from each immunised animal were added to the wells, with or without an HBsAg-derived peptide (IPQSLDSWWTSL). For all cell concentrations tested with the HBsAg peptide, control wells (without the HBsAg peptide) with the same number of splenocytes were included in the assay. Half a million target cells (splenocytes from naive BALB/c mice) were added to all wells. Following incubation, splenocytes were removed and any secreted IFN was detected following incubation with a biotinylated rat anti-mouse IFN antibody followed by a streptavidin-alkaline phosphatase conjugate and subsequent colour development with BCIP (5-bromo-4-chloro-3-indolul phosphate) and NBT (nitroblu tetrazolium). Results were expressed as the number of IFN -secreting cells (spot-forming cells; SFC) per million splenocytes. For each cell concentration tested, the background number of SFC in the relevant control wells was subtracted from the number of SFC in wells incubated with the HBsAg peptide to give the number of peptide-specific SFC/well. For each sample, the number of SFC per million splenocytes was then calculated from the wells with the highest concentration of cells (31,000 splenocytes per well) that gave rise to 50-200 peptide-specific SFC/well, or the lowest cell concentration tested (31,000 s plenocytes per well) where all values were greater than 200 SFC/well.

### Results

Groups of animals injected with four doses of pSG2.HBs, given two weeks apart, developed low, but detectable, CD8+ T cell responses as measured in a lysis assay or by an ELISPOT assay (40-100 SFC/10⁶ splenocytes). Groups of animals injected with four doses of MVA.HBs developed relatively strong CD8+ T cell responses as detected by both assays (80-150 SFC/10⁶ splenocytes). However, all animals that were primed by two immunisations with pSG2.HBs, and boosted twice with MVA.HBs, developed very strong CD8+ T cell responses (150-250 SFC/10⁶ splenocytes).

### Cytotoxicity Assay—Results

Lysis of P815 target cells by in vitro-restimulated splenocytes pooled from five animals at a ratio of effector:target (E:T) cells of 33:1, are summarised in Table 1.

TABLE 1
Induction of Specific Cytolytic Responses

| | “Day 1” (5 animals) | | “Day 2” (5 animals) | |
|---|---|---|---|---|
| Treatment group | Males | Females | Males | Females |
| 1 (control) | 16.7 | 3.4 | 0 | 0 |
| 2 (plasmid DNA) | 33.5 | 18.2 | 1.5 | 0.3 |
| 3 (MVA) | 28.4 | 27.0 | 0.4 | 41.2 |
| 4 (plasmid DNA + | 73.0 | 20.7 | 7.3 | 60.4 |
| MVA) | | | | |

No CTL response (other than a borderline response in one male group) was seen in control animals (group 1). A moderate CTL response was detected in one group of male animals and one group of female animals receiving plasmid DNA alone (group 2). Both groups of female animals but only one of the male groups receiving MVA.HBS alone (group 3) mounted a CTL response. Similarly, both groups of female animals and only one male group receiving the DNA prime:MVA boost regimen (group 4) mounted a CTL response. The responses seen in two of these groups were, however, substantially higher than those seen in animals immunised with DNA or MVA alone.

### ELISPOT Results

No response was detected in control animals (group 1). Animals receiving plasmid DNA alone (group 2) had detectable, but low levels of SFC (generally <100 SFC/10⁶ splenocytes). Immunisation with MVA.HBs alone (group 3) gave similar responses, with one group of females having a slightly higher response. In contrast, both male and female animals receiving the DNA prime:MVA boost regimen (group 4) had high levels of peptide-specific IFN-γ-secreting T cells.

TABLE 2
Induction of Specific IFN-γ-secreting T Cells

| | “Day 1” (5 animals) | | “Day 2” (5 animals) | |
|---|---|---|---|---|
| Treatment group | Males | Females | Males | Females |
| 1 (control) | 3 | 13 | 3 | 10 |
| 2 (plasmid DNA) | 107 | 42 | 62 | 77 |
| 3 (MVA) | 96 | 118 | 63 | 159 |
| 4 (plasmid DNA + | 245 | 262 | 255 | 169 |
| MVA) | | | | |

In summary, priming of BALB/c mice with plasmid pSG2.HBs and boosting with MVA.HBs resulted in the induction of both CTL responses and IFN-γ-secreting T cells (as measured by the ⁵¹Cr lysis and ELISPOT assays, respectively). The prime-boost regimen induced stronger responses than immunisation with plasmid DNA alone. Administration of MVA.HBs alone resulted in similar levels of CTL but lower levels of IFN-γ-secreting T cells, compared with the prime-boost regimen.

These results demonstrate the effective delivery and presentation, in vivo, of the HBsAg antigen by both the plasmid and MVA delivery systems, as well as the enhanced immunogenicity of the DNA prime:MVA boost regimen in the mouse.

### EXAMPLE 4

### Patient Studies—Phase I Clinical Studies of an HBV Immunotherapeutic in Healthy Individuals

Two phase I studies were conducted. In the first study, 18 healthy male subjects in the United Kingdom were divided into two groups of nine (Group A and Group B). In Group A, six subjects received four injections of 5×10⁷ pfu MVA.HBs i.d. and three subjects received two placebo injections followed by two injections of 5×10⁷pfu MVA.HBs i.d. All injections were administered at three week intervals. In Group B, six subjects received two injections of 1 mg pSG2.HBs i.m., followed by two doses of 5×10⁷ pfu MVA.HBs i.d., while the other three subjects received two placebo injections, followed by two injections of 5×10⁷ pfu MVA.HBs i.d. Injections were administered at three week intervals.

There were no significant changes seen in vital signs (e.g., blood pressure, heart rate, temperature and respiratory rate) in any subject on trial. No local side effects were seen after pSG2.HBs injection. Mild to moderate injection site reactions were seen in all subjects dosed with MVA.HBs. These reactions including erythema, swelling, flaking, and tenderness. One subject reported aching joints and tiredness two days after the first injection of MVA and one reported tingling of the tip of his tongue on the day of the second injection, both of which were considered possibly or probably related to trial drug. All other treatment-related adverse events were injection site-related.

Although some changes were observed in liver function tests (LFTs), in seven subjects (one on placebo and six on active treatment), only two subjects did not complete the full course of treatment due to raised LFTs. A third subject in Group A showed an increase in g-glutamyl transferase (GGT) above the normal range and a mild increase in alanine transferase (ALT) prior to receiving the second injection. Values returned to normal after this and the third injection but GGT increased to more than twice the upper limit of normal prior to the fourth injection, which was therefore not given. This subject was referred to a hepatologist, who found no abnormality other than the enzyme values. One subject (Subject 11) in Group B had GGT levels around 50% above the upper limit of normal on screening, which values increased to more than twice the upper limit of normal after the first and second injections, accompanied by abnormal ALT values. The third and fourth doses were withheld in this subject. All values returned to normal 3 weeks post the last injection except the GGT in Subject 11 which returned to pre-dose values.

These changes in LFTs did not show a clear pattern or temporal relationship to trial treatment. Some subjects entered the trial with abnormal values, probably related to lifestyle, and, therefore, were probably not suitable subjects. The protocol did not prohibit alcohol intake during the trial period and the subjects were not institutionalised or tested for blood alcohol levels during the study. For Group A, the third and fourth injections and for Group B, the second and third injections spanned the Christmas/New Year period. It is possible, therefore, that increased alcohol intake may have confounded the interpretation of these LFT changes and no firm conclusion can be drawn in terms of relationship to trial treatment.

In Group A, immunisation induced IFN-gamma-secreting HBs-specific T cell responses in two subjects who received four active MVA injections. In Group B, an HBs-specific T-cell response was seen in two subjects who received two placebo then two active MVA injections and in one subject who received two DNA then two MVA injections. The level of T cell responses was moderate and peaked seven days post-MVA inoculation. Responses against HBs-derived peptide pools correlated with detection of T cell responses against HBs antigen and HLA-A2 peptide pools. No HBs-specific antibody response was observed. All volunteers who received MVA developed very high titres of MVA-specific antibodies.

A second phase I study was carried out in eight healthy volunteer subjects in The Gambia. Five subjects were treated with 1 mg pSG2.HBs twice. Three others were treated with 5×10⁷ pfu MVA.HBs twice. No significant abnormalities were seen in laboratory safety tests after dosing. Local side effects were similar to those seen in the first study, described above, although erythema was less noticeable, and shiny plaques were also observed in pigmented skin. Two subjects experienced itching and one subject experienced scaling of skin at the DNA injection site. All subjects experienced local side effects including hardness, scaling and shiny plaque formation at MVA injection sites. One subject reported feeling feverish 5 hours after DNA injection and one reported headache 5 days after DNA injection. Other minor side effects were considered unrelated to trial treatment. HBs-specific immune responses were seen in 4 of 5 volunteers dosed with DNA alone and in 3 of 3 volunteers dosed with MVA alone, the size of the MVA response being approximately double that response observed for the DNA plasmid. All of these volunteers had anti-HBs antibodies present at baseline (indicating past, resolved, hepatitis B infection). There was no increase in anti-HBs antibody levels after DNA immunisation, but two of three MVA-treated volunteers showed an increase in anti-HBs after MVA immunisation. It is postulated that the greater immune response seen in the Gambian trial population relative to the U.K. volunteers was due to this prior infection.

The Phase I study has shown that two doses of 1 mg pSG2.HBs i.m., followed by two doses of 5×10⁷ pfu MVA.HBs i.d., at three week intervals, or four doses of 5×10⁷ pfu MVA.HBs i.d., at three week intervals, were well tolerated in healthy volunteers.

### EXAMPLE 5

### Clinical Determination of Optimum Dosing Regimens for an HBV Immunotherapeutic Involving Plasmid and MVA Delivery

Following on from the phase I study (described herein), three different dosage regimens were tested in patients with chronic HBV hepatitis to determine the optimum dosage regimen for treatment. The particular objectives of this study were:

1) to assess the tolerability of three different dosing regimens of a hepatitis immunotherapeutic in patients with HBeAg-positive, chronic hepatitis B; and

2) to determine the cellular immune response induced by these three dosing regimens.

### Materials and Methods

### PART ONE—Investigation of Dosing Regimens:

Three different dosing regimens of pSG2.HBs and MVA.HBs were investigated in an open, non-randomised, rising-dose fashion. Treatment regimens were administered as follows:

Group 1: Two doses of 1 mg pSG2.HBs i.m., followed by two doses of 5×10⁷ pfu MVA.HBs i.d.

Group 2: Two doses of 2 mg pSG2.HBs i.m., followed by two doses of 1.5×10⁸ pfu MVA.HBs i.d.

Group 3: Two doses of 2 mg pSG2.HBs i.m., followed by two doses of 5×10⁸ pfu MVA.HBs i.d.

In each regimen, the interval between immunizations was three weeks (e.g., administration of doses at Weeks 0, 3, 6 and 9).

### PART TWO—Efficacy of Dosing Regimens

Fifty-four Patients were randomly assigned to one of the three treatment groups. Treatment regimens for each group were as follows:

Group A: Two doses of 2 mg pSG2.HBs i.m., followed by two doses of 5×10⁸ pfu MVA.HBs i.d., with three week intervals between doses (e.g., dosing at Weeks 0, 3, 6 and 9).

Group B: 100 mg of lamivudine were administered daily for 14 weeks. In addition, two doses of 2 mg pSG2.HBs i.m., followed by two doses of 5×10⁸ pfu MVA.HBs i.d., were administered with three week intervals between doses (e.g., dosing at Weeks 0, 3, 6 and 9). Administration of lamivudine commenced 4 weeks prior to the first immunotherapeutic dose (immunotherapeutic dosing at Weeks 4, 7, 10 and 13).

Group C: 100 mg of lamivudine were administered daily for 14 weeks. The treatment phase visit schedule for the patients in groups A, B and C of Part 2 were as shown in Table 7.

TABLE 7
Dosage Regimen for Groups A, B and C.

| Wk | S | 0 | 1 | 2 | 3 | 4 | 5 | 6 | 7 | 8 | 9 | 10 | 11 | 12 | 13 | 14 | 18 |
|---|---|---|---|---|---|---|---|---|---|---|---|---|---|---|---|---|---|
| A | S | D | v | | D | v | | M | v | | M | v | | | | v | |
| B | S | L | | | | D | v | | D | v | | M | v | | M | v | v |
| C | S | L | | | | v | | | v | | | v | | | | v | v |

Key:
S = Screening carried out at approximately week - 14
D = pSG2.HBs immunisation (2 mg)
M = MVA.HBs immunisation (5 × 10⁸ pfu)
L = Start Lamivudine therapy (100 mg/day)
v = Non-immunisation visit

### Assessment of Safety and Tolerability

### Definitions

An adverse event (AE) is any undesirable medical experience or change of an existing condition that occurs during or after administration of an investigational agent, whether or not it is considered related to the trial. Abnormal laboratory findings considered by the Principal Investigator to be clinically significant, e.g., those that are unusual or unusually severe for the population being studied, were considered to be adverse events. In addition, any unusual or extreme injection site reactions (such as scabbing, abscesses or ulcerations) were recorded as AEs, as were any injection site reaction that persists for more than 7 days.

A serious adverse event (SAE) is any experience that causes a significant hazard to the patient and includes any event that is fatal, life-threatening (places the patient at immediate risk of death), and/or requires or prolongs hospitalisation, as well as any event that is significantly or permanently disabling, constitutes a (new) malignancy, or is a congenital abnormality/birth defect in the offspring of a patient who was participating in the trial at the time of conception or during the pregnancy of the mother.

### Assessment of Injection Site Reactions

Injection sites were assessed at 30 minutes (Groups A and B) or 2 hours (Groups 1, 2 and 3) after immunisation, as well as 7 days after immunisation, and 5 weeks after the final immunisation. Injection Site Reactions were separately classified according to the severity of the following indications: swelling, pain, erythema, itch and ulceration.

Injection site reactions were designated as either mild, moderate or severe based on size (erythema and swelling) or clinical judgement (itchiness, ulceration and pain). For assessment of erythema and swelling, sizes of less than 1 cm, 1-3 cm, and greater than 3 cm were designated as mild, moderate and severe, respectively.

### Results

Eighty-seven total treatment-related adverse events (AEs) were recorded over the course of the treatment phase of the study, of which sixty-one of these events were related to the injection site (Table 3). As discussed herein, the majority of injection site reactions were comparatively mild. Furthermore, systemic adverse events were generally associated with an immune response, such as flu-like symptoms.

Only three serious adverse effects (SAEs) were reported during the 14-week course of treatment, of which one was unrelated to the treatment. The other two SAEs were incidents of elevated aminotransferase (ALT) activity, and required hospitalisation of the patient for observation. Increased ALT can be associated with viral clearance from the liver, and at least two of these SAEs are thought to be implicated in a clinical response due to the treatment program.

TABLE 3
Summary of Adverse Events (AEs) Following Various Treatment Regimens

| | Part 1 | | | Part 2 | | | |
|---|---|---|---|---|---|---|---|
| | Gp 1 | Gp 2 | Gp 3 | Gp A | Gp B | Gp C | |
| | (N = 7) | (N = 6) | (N = 6) | (N = 21) | (N = 22) | (N = 11) | Total |
| Total No. AEs | 2 | 2 | 18 | 43 | 41 | 4 | 110 |
| Treatment related | 2 | 1 | 17 | 38 | 28 | 1 | 87 |
| At injection site | 0 | 0 | 12 | 34 | 15 | N/A | 61 |
| SAEs | 1 | 0 | 0 | 2 | 0 | 0 | 3 |

Reactions at the injection site showed that the DNA and MVA immunotherapeutics were generally safe and well tolerated at dosages up to, and including, 5×10⁸ pfu of MVA.HBs. The majority of injection site reactions were mild (72%), and only 2% of injection sites were classified as having severe injection site reactions in this study (Table 4). Notably, a greater number of local and systemic events that were consistent with the induction of an immune response to therapy, were also observed in the group that received the highest dose. As a result, the highest dosage regimen (e.g., two doses of 2 mg pSG2.HBs followed by two doses of 5×10⁸ pfu MVA.HBs.) was chosen for use in PART TWO.

TABLE 4
Severity of Injection Site Reactions

| | Part 1 | | | Part 2 | | | |
|---|---|---|---|---|---|---|---|
| | Gp 1 | Gp 2 | Gp 3 | Gp A | Gp B | Total | |
| | (N = 7) | (N = 6) | (N = 6) | (N = 21) | (N = 22) | (N = 62) | |
| No. Pt with ≧1 | 5 | 4 | 6 | 20 | 19 | 54 | (87%) |
| Mild | 9 | 11 | 9 | 83 | 92 | 204 | (73%) |
| Moderate | 5 | 0 | 6 | 40 | 20 | 71 | (25%) |
| Severe | 1 | 1 | 4 | 0 | 0 | 6 | (2%) |
| TOTAL | 15 | 12 | 19 | 123 | 112 | 281 | |

The predominant injection site reactions consisted of mild or moderate swelling or erythema, with lower incidences of mild itch, pain and ulceration (Table 5). The number of moderate injection site reactions decreased with the second MVA.HBs injection, consistent with the patients becoming more tolerant to the MVA vaccination (Table 6).

Collectively, the data obtained in this study indicated that the tested therapeutic vaccination regimens involving pSG2.HBs and MVA.HBs produced side effects that were generally limited to the injection site. Combination with lamivudine did not result in an increase in side effects (compare Group A and Group B in Tables 1-4). Therefore, the present invention provides a therapy for HBV that is better tolerated than currently available immunotherapeutics, such as interferon.

TABLE 5
Injection Site Reactions Categorized by Reaction Type

| | | Group 1 | Group 2 | Group 3 | Group A | Group B | Total |
|---|---|---|---|---|---|---|---|
| Reaction | Assessment | (N = 7) | (N = 6) | (N = 6) | (N = 21 | (N = 22) | (N = 62) |
| Erythema | Mild | 4 | 5 | 5 | 20 | 27 | 61 |
| | Moderate | 3 | 0 | 3 | 19 | 6 | 31 |
| | Severe | 0 | 0 | 1 | 0 | 0 | 1 |
| Swelling | Mild | 3 | 2 | 4 | 20 | 23 | 51 |
| | Moderate | 1 | 0 | 2 | 14 | 7 | 25 |
| | Severe | 1 | 0 | 2 | 0 | 0 | 3 |
| Itch | Mild | 0 | 1 | 0 | 15 | 18 | 34 |
| | Moderate | 0 | 0 | 0 | 5 | 5 | 10 |
| | Severe | 0 | 0 | 0 | 0 | 0 | 0 |
| Pain | Mild | 2 | 1 | 0 | 22 | 22 | 47 |
| | Moderate | 1 | 0 | 1 | 2 | 2 | 6 |
| | Severe | 0 | 1 | 1 | 0 | 0 | 2 |
| Ulceration | Mild | 0 | 2 | 0 | 6 | 2 | 10 |
| | Moderate | 0 | 0 | 0 | 0 | 0 | 0 |
| | Severe | 0 | 0 | 0 | 0 | 0 | 0 |
| | TOTAL | 15 | 12 | 19 | 123 | 112 | 281 |

TABLE 6
Injection Site Reactions Categorized by Immunization

| | | Group 1 | Group 2 | Group 3 | Group A | Group B | Total |
|---|---|---|---|---|---|---|---|
| Immunization | Assessment | (N = 7) | (N = 6) | (N = 6) | (N = 21 | (N = 22) | (N = 62) |
| PSG2.HBs | Mild | 0 | 0 | 0 | 6 | 5 | 11 |
| | Moderate | 0 | 0 | 0 | 1 | 0 | 1 |
| | Severe | 0 | 0 | 0 | 0 | 0 | 0 |
| PSG2.HBs | Mild | 0 | 0 | 0 | 4 | 3 | 7 |
| | Moderate | 0 | 0 | 0 | 0 | 0 | 0 |
| | Severe | 0 | 0 | 0 | 0 | 0 | 0 |
| MVA.HBs | Mild | 5 | 7 | 5 | 26 | 45 | 88 |
| | Moderate | 4 | 0 | 6 | 31 | 16 | 57 |
| | Severe | 1 | 0 | 1 | 0 | 0 | 2 |
| MVA.HBs | Mild | 4 | 4 | 4 | 47 | 39 | 100 |
| | Moderate | 1 | 0 | 0 | 8 | 4 | 11 |
| | Severe | 0 | 1 | 3 | 0 | 0 | 4 |
| | TOTAL | 15 | 12 | 19 | 123 | 112 | 281 |

### EXAMPLE 6

### Clinical Determination of the Efficacy of an HBV Immunotherapeutic Involving Plasmid and MVA Delivery Relative to a Known HBV Treatment

Lamivudine (3TC) is a nucleoside analogue inhibitor of reverse transcriptase activity, which was initially developed as an anti-HIV agent. Lamivudine can be given orally and shows two modes of viral suppression. First, the active triphosphate metabolite mimics deoxycytidine triphosphate and is incorporated into newly synthesised HBV DNA, leading to chain termination. Second, the active form shows competitive inhibition of reverse transcriptase activity.

Studies of lamivudine in patients with chronic HBV infection have shown that treatment with lamivudine results in a rapid decrease in the plasma levels of HBV DNA. However, these levels return to baseline on cessation of therapy. Lamivudine is generally well tolerated and is licensed for the treatment of adults with chronic hepatitis B associated with HBV replication and active liver disease. One downside of extended lamivudine monotherapy is the development of mutations in the HBV genome and viral resistance.

The efficacy of the HBV immunotherapeutic of the present invention, involving DNA plasmid/MVA delivery, was compared with that of a lamivudine/therapeutic HBV immunotherapeutic combination, and treatment with lamivudine alone. The antiviral efficacy of each treatment was measured by assessing seroconversion rates, plasma HBV DNA load and levels of the liver enzymes, alanine transferase (ALT) and aspartate transferase (AST), throughout the first 14/18 weeks of treatment and at 6, 9 and 12 months after the start of treatment. Tolerability and immune response were assessed at intervals throughout the treatment period.

### Materials and Methods

### ELISPOT Assays

ELISPOT Assays were carried out by Cellular Technology Limited (CTL Laboratory LLC. 10515 Carnegie Ave., Suite 503 Cleveland Ohio 44106, USA) using the following assay conditions:

| | |
|---|---|
| Antigen preparation | Fresh |
| Thawing method | Warm + DNase |
| Counting method | Guava ViaCount ® |
| Cell preparation | PBMC thawed and used immediately |
| No. cells per well in ELISPOT | 3 × 10⁵ |
| Length of assay | 24 hours |
| Type of spot counter | CTL Immunospot Reader |

The following peptides were pooled and used as antigens:

| | Disease/ | | Peptide |
|---|---|---|---|
| Antigen | Model | Antigen | Sequence |
| HBS 1 | Hepatitis B | PreS2 + S ayw | MQWNSTTFHQTLQDP |
| | | | (SEQ ID NO: 16) |
| HBS 2 | Hepatitis B | PreS2 + S ayw | TFHQTLQDPRVRGLY |
| | | | (SEQ ID NO: 17) |
| HBS 3 | Hepatitis B | PreS2 + S ayw | QDPRVRGLYFPAGGS |
| | | | (SEQ ID NO: 18) |
| HBS 4 | Hepatitis B | PreS2 + S ayw | GLYFPAGGSSSGTVN |
| | | | (SEQ ID NO: 19) |
| HBS 5 | Hepatitis B | PreS2 + S ayw | GGSSSGTVNPVLTTA |
| | | | (SEQ ID NO: 20) |
| HBS 6 | Hepatitis B | PreS2 + S ayw | TVNPVLTTASPLSSI |
| | | | (SEQ ID NO: 21) |
| HBS 7 | Hepatitis B | PreS2 + S ayw | TTASPLSSIFSRIGD |
| | | | (SEQ ID NO: 22) |
| HBS 8 | Hepatitis B | PreS2 + S ayw | SSIFSRIGDPALNME |
| | | | (SEQ ID NO: 23) |
| HBS 9 | Hepatitis B | PreS2 + S ayw | IGDPALNMENITSGF |
| | | | (SEQ ID NO: 24) |
| HBS 10 | Hepatitis B | PreS2 + S ayw | NMENITSGFLGPLLV |
| | | | (SEQ ID NO: 25) |
| HBS 11 | Hepatitis B | PreS2 + S ayw | SGFLGPLLVLQAGFF |
| | | | (SEQ ID NO: 26) |
| HBS 12 | Hepatitis B | PreS2 + S ayw | LLVLQAGFFLLTRIL |
| | | | (SEQ ID NO: 27) |
| HBS 13 | Hepatitis B | PreS2 + S ayw | GFFLLTRILTIPQSL |
| | | | (SEQ ID NO: 28) |
| HBS 14 | Hepatitis B | PreS2 + S ayw | RILTIPQSLDSWWTS |
| | | | (SEQ ID NO: 29) |
| HBS 15 | Hepatitis B | PreS2 + S ayw | QSLDSWWTSLNFLGG |
| | | | (SEQ ID NO: 30) |
| HBS 16 | Hepatitis B | PreS2 + S ayw | WTSLNFLGGTTVCLG |
| | | | (SEQ ID NO: 31) |
| HBS 17 | Hepatitis B | PreS2 + S ayw | LGGTTVCLGQNSQSP |
| | | | (SEQ ID NO: 32) |
| HBS 18 | Hepatitis B | PreS2 + S ayw | CLGQNSQSPTSNHSP |
| | | | (SEQ ID NO: 33) |
| HBS 19 | Hepatitis B | PreS2 + S ayw | QSPTSNHSPTSCPPT |
| | | | (SEQ ID NO: 34) |
| HBS 20 | Hepatitis B | PreS2 + S ayw | HSPTSCPPTCPGYRW |
| | | | (SEQ ID NO: 35) |
| HBS 21 | Hepatitis B | PreS2 + S ayw | PPTCPGYRWMCLRRF |
| | | | (SEQ ID NO: 36) |
| HBS 22 | Hepatitis B | PreS2 + S ayw | YRWMCLRRFIIFLFI |
| | | | (SEQ ID NO: 37) |
| HBS 23 | Hepatitis B | PreS2 + S ayw | RRFIIFLFILLLCLI |
| | | | (SEQ ID NO: 38) |
| HBS 24 | Hepatitis B | PreS2 + S ayw | LFILLLCLIFLLVLL |
| | | | (SEQ ID NO: 39) |
| HBS 25 | Hepatitis B | PreS2 + S ayw | CLIFLLVLLDYQGML |
| | | | (SEQ ID NO: 40) |
| HBS 26 | Hepatitis B | PreS2 + S ayw | VLLDYQGMLPVCPLI |
| | | | (SEQ ID NO: 41) |
| HBS 27 | Hepatitis B | PreS2 + S ayw | GMLPVCPLIPGSSTT |
| | | | (SEQ ID NO: 42) |
| HBS 28 | Hepatitis B | PreS2 + S ayw | PLIPGSSTTSTGPCR |
| | | | (SEQ ID NO: 43) |
| HBS 29 | Hepatitis B | PreS2 + S ayw | STTSTGPCRTCMTTA |
| | | | (SEQ ID NO: 44) |
| HBS 30 | Hepatitis B | PreS2 + S ayw | PCRTCMTTAQGTSMY |
| | | | (SEQ ID NO: 45) |
| HBS 31 | Hepatitis B | PreS2 + S ayw | TTAQGTSMYPSCCCT |
| | | | (SEQ ID NO: 46) |
| HBS 32 | Hepatitis B | PreS2 + S ayw | SMYPSCCCTKPSDGN |
| | | | (SEQ ID NO: 47) |
| HBS 33 | Hepatitis B | PreS2 + S ayw | CCTKPSDGNCTCIPI |
| | | | (SEQ ID NO: 48) |
| HBS 34 | Hepatitis B | PreS2 + S ayw | DGNCTCIPIPSSWAF |
| | | | (SEQ ID NO: 49) |
| HBS 35 | Hepatitis B | PreS2 + S ayw | IPIPSSWAFGKFLWE |
| | | | (SEQ ID NO: 50) |
| HBS 36 | Hepatitis B | PreS2 + S ayw | WAFGKFLWEWASARF |
| | | | (SEQ ID NO: 51) |
| HBS 37 | Hepatitis B | PreS2 + S ayw | LWEWASARFSWLSLL |
| | | | (SEQ ID NO: 52) |
| HBS 38 | Hepatitis B | PreS2 + S ayw | ARFSWLSLLVPFVQW |
| | | | (SEQ ID NO: 53) |
| HBS 39 | Hepatitis B | PreS2 + S ayw | SLLVPFVQWFVGLSP |
| | | | (SEQ ID NO: 54) |
| HBS 40 | Hepatitis B | PreS2 + S ayw | VQWFVGLSPTVWLSV |
| | | | (SEQ ID NO: 55) |
| HBS 41 | Hepatitis B | PreS2 + S ayw | LSPTVWLSVIWMMWY |
| | | | (SEQ ID NO: 56) |
| HBS 42 | Hepatitis B | PreS2 + S ayw | LSVIWMMWYWGPSLY |
| | | | (SEQ ID NO: 57) |
| HBS 43 | Hepatitis B | PreS2 + S ayw | MWYWGPSLYSILSPF |
| | | | (SEQ ID NO: 58) |
| HBS 44 | Hepatitis B | PreS2 + S ayw | SLYSILSPFLPLLPI |
| | | | (SEQ ID NO: 59) |
| HBS 45 | Hepatitis B | PreS2 + S ayw | SPFLPLLPIFFCLWV |
| | | | (SEQ ID NO: 60) |
| HBS 46 | Hepatitis B | PreS2 + S ayw | FLPLLPIFFCLWVYI |
| | | | (SEQ ID NO: 61) |

The HBs series of peptides are arranged into 4 distinct pools of peptides, each containing up to 12 total peptides:

Pool No.

| 1 | 2 | 3 | 4 |
|---|---|---|---|
| 1 | 12 | 23 | 36 |
| 2 | 13 | 25 | 37 |
| 3 | 14 | 26 | 38 |
| 4 | 15 | 27 | 39 |
| 5 | 16 | 28 | 40 |
| 6 | 17 | 29 | 41 |
| 7 | 18 | 30 | 42 |
| 8 | 19 | 31 | 43 |
| 9 | 20 | 32 | 44 |
| 10 | 21 | 33 | 45 |
| 11 | 22 | 34 | 46 |
| | | 35 | |

The entire HbsAg protein was also used as an antigen.

An ELISPOT response to a peptide pool (or HbsAg protein) was considered to have occurred when the mean +/− standard deviation for the peptide pool, or HbsAg protein, was greater than the mean +3 standard deviations of that patient's negative control.

### Liver Enzyme (ALT) Assays

Liver enzyme assays were carried out by the hospital analytical laboratories at each study site, according to local procedures.

### Viral Load

Viral load measurements were carried out as follows:

Groups 1-3: An Amplicor HBV Monitor (Roche) was used to measure viral load, according to the manufacturer's instructions:

Sensitivity: 200 copies/mL

Linear range: 1000 to 4×10⁷ copies/mL

Groups A, B, C: The COBAS Taqman system (Roche) was used according to the manufacturer's instructions:

Sensitivity: 35 copies/mL

Linear range: 169-6.4×10⁸ copies/mL

All viraemias above the linear range were reported as “Above maximum linear range”.

### HBeAg, HBsAg, Anti-HBe and Anti-HBs Serotyping

Serotyping was performed using a VITROS ECi Immunodiagnostic System.

### Results: Efficacy

The responses to treatment for each treatment group, i.e., Groups 1-3 (see Example 5), Group A (immunotherapeutic), Group B (immunotherapeutic+lamivudine) and Group C (lamivudine alone), are summarised in Table 8. Significantly, 2 out of 19 patients in Groups 1-3, and 3 out of 21 patients in Group A, seroconverted to HBe (became HBeAG negative and anti HBe positive) by week 14 (Table 9). None of the 11 patients in the lamivudine control group (Group C) had seroconverted after 14 weeks of therapy with lamivudine alone.

TABLE 8
Treatment Phase Summary Response

| | Part 1 | Gp A | Gp B | Gp C |
|---|---|---|---|---|
| | (n = 19) | (n = 21) | (n = 22) | (n = 11) |
| HBeAg Loss | 4* | 5 | 3 | 1 |
| HBeAg seroconversion | 2 | 3 | 0 | 0 |
| HBsAg Loss | 0 | 0 | 0 | 1 |
| HBsAg seroconversion | 0 | 0 | 0 | 0 |
| HBV DNA <1.5 log | 1 | 5 | 18 | 9 |
| ALT Normalized | 4 | 1 | 4 | 2 |
| Immunological response | N/A | 6 | 9 | 5 |

Table 8 shows that patients in each group demonstrated therapeutic responses to treatment, including drop in viral load, normalisation of liver enzyme (ALT) levels and immunological responses to HBs antigens (as determined by ELISPOT assays of CD8+ interferon-gamma secreting T cells; ELISPOT data was not available for Groups 1-3). Tables 9 and 10 show that anti-HBe seroconversion was also observed for one patient in PART ONE (Groups 1-3), 52 weeks after the start of the study. PART TWO (Groups A-C) are still currently under observation. Tables 11-14 indicate the presence or absence of various treatment responses in individual patients.

TABLE 9
HBeAg Clearance and anti-HBe Seroconversion

| | | Part 1 | | | | |
|---|---|---|---|---|---|---|
| | | (Gps 1-3 | | Part 2 | | |
| | | combined) | Group A | Group B | Group C | Total |
| Week 0 | n | 19 | 21 | 22 | 11 | 73 |
| | HBeAg+ | 19** | 21 | 22 | 11 | 73 |
| Week | HBeAg+ | 13 | 14 | 13 | 8 | 48 |
| 14/18 | HBeAg− | 4 (21%) | 5 (24%) | 3 (14%) | 1 (9%) | 13 |
| | Anti Hbe+ | 2 | 4* | 0 | 0 | 6 |
| | Seroconversion | 2 (11%) | 3 (14%) | 0 | 0 | 5 |
| Week 52 | HBeAg+ | 14 | — | — | — | 14 |
| | HBeAg− | 2 (11%) | — | — | — | 2 |
| | Anti Hbe+ | 1 | — | — | — | 1 |
| | Seroconversion | 1 (5%) | — | — | — | 1 |

*One patient became anti-HBe+ but did not lose HBeAg.

TABLE 10
HBsAg Clearance and anti-HBs Seroconversion

| | | Part 1 | | | | |
|---|---|---|---|---|---|---|
| | | (Gps 1-3 | | Part 2 | | |
| | | combined) | Group A | Group B | Group C | Total |
| Week 0 | n | 19 | 21 | 22 | 11 | 73 |
| | HBsAg+ | 19 | 21 | 22 | 11 | 73 |
| Week | HBsAg+ | 17 | 18 | 16 | 8 | 59 |
| 14/18 | HBsAg− | 0 | 0 | 0 | 1 (9%) | 1 |
| | Anti Hbs+ | 1 | 1 | 0 | 1 | 3 |
| | Seroconversion | 0 | 0 | 0 | 0 | 0 |
| Week 52 | n | 16 | 0 | 0 | 0 | 16 |
| | HBsAg+ | 16 | — | — | — | 16 |
| | HBsAg− | 0 | — | — | — | 0 |
| | Anti Hbs+ | 0 | — | — | — | 0 |
| | Seroconversion | 0 | — | — | — | 0 |

TABLE 11
Summary - Treatment Responses for Individuals from Combined Groups 1-3

| | Week 14 | | | | Week 52 | | | |
|---|---|---|---|---|---|---|---|---|
| | | | HBV DNA | | | | HBV DNA | |
| | | Anti | drop >1.5 | ALT | HBeAg− | Anti | drop >1.5 | ALT |
| Pt | HBeAg− | HBe+ | log | norm | loss | HBe+ | log | norm |
| 101 | | | | X | | | X* | X |
| 102 | X | X | X | X | | | | X |
| 103 | X | | | | | | | |
| 104 | | | | | X | | X | |
| 107 | X | | | X | X | X | X | X |
| 202 | X | X | | | | | | |
| 304 | | | | | | | | |

TABLE 12
Summary of Treatment Responses for Individuals from Group A

| | HBeAg | Anti | HBV DNA | ALT | Immune |
|---|---|---|---|---|---|
| Patient | loss | HBe+ | drop >1.5 log | norm | response |
| 401 | | | | | X |
| 405 | | | X | | |
| 407 | X | X | X | | X |
| 408 | X | | | | |
| 409 | | | | X | |
| 415 | X | X | X | | |
| 416 | X | | | | X |
| 417 | | X | | | |
| 418 | | | | | X |
| 419 | | | | | X |
| 421 | X | X | X | | X |

TABLE 13
Summary of Treatment Responses for Individuals from Group B

| | HBeAg | Anti | HBV DNA | ALT | Immune |
|---|---|---|---|---|---|
| Patient | loss | HBe+ | drop >1.5 log | norm | response |
| 401 | | | | | X |
| 405 | | | X | | |
| 407 | X | X | X | | X |
| 408 | X | | | | |
| 409 | | | | X | |
| 415 | X | X | X | | |
| 416 | X | | | | X |
| 417 | | X | | | |
| 418 | | | | | X |
| 419 | | | | | X |
| 421 | X | X | X | | X |

TABLE 14
Summary of Treatment Responses for Individuals from Group C

| | HBeAg | Anti | HBV DNA | ALT | Immune |
|---|---|---|---|---|---|
| Patient | loss | HBe+ | drop >1.5 log | norm | response |
| 601 | | | X | X | |
| 604 | | | X | | X |
| 605 | X | | X | | X |
| 606 | | | X | X | |
| 607 | | | X | | X |
| 608 | | | X | | X |
| 610 | | | X | | X |

Observed immunological responses were classed into the following groups: “baseline”—an immune response (as defined above) was detected prior to commencement of treatment ( i.e., week 0 for Groups 1-3 and Group A, and week 4 for Groups B and C). Said immune response subsequently was unchanged or reduced during the course of the study; “boosted”—an immune response was detected prior to commencement of treatment as discussed above, and increased with treatment over the course of the study; “de novo”—an immune response was not present at the start of the study, but appeared following therapy. Group A (immunotherapeutic alone) included 3 baseline immunological responses, 2 boosted responses (i.e., 2/5 baseline responses were boosted by the therapy) and 4 de novo responses (i.e., 4/16 baseline non-responders gained an immune response following therapy) (Table 12). Group B (immunotherapeutic+lamivudine) included 3 baseline immunological responses, 4 boosted responses (i.e., 4/7 baseline responses were boosted by the therapy) and 5 de novo responses (i.e., 5/15 baseline non-responders gained an immune response following therapy) (Table 13). Group C (lamivudine alone) included 3 baseline immunological responses, 5 boosted responses (i.e., 5/8 baseline responses were boosted by lamivudine), but no de novo responses (0/3 baseline non-responders gained an immune response following lamivudine therapy) (Table 14).

These data demonstrate that the immunotherapeutic of the present invention (e.g., an HBV immunotherapeutic involving DNA plasmid and recombinant MVA delivery) is able to stimulate immune responses in previously unresponsive HBV patients. In contrast, treatment with lamivudine alone was unable to stimulate immune responses in previously unresponsive HBV patients. The immunotherapeutic of the present invention was also able to produce up to 14% seroconversion (Group A; Table 12) by a series of four injections over only 14 weeks. No seroconversion was observed over the 14 weeks of Group C (lamivudine only; Table 14). The immunotherapeutic, therefore, represents a viable approach to therapy of chronic HBV and provides further proof of the efficacy of “prime-boost” immunotherapeutics in the clinic.

### EXAMPLE 7

### Elevated Aminotransferase Activity that is Temporally Linked to Immunizations is Associated with Seroconversion

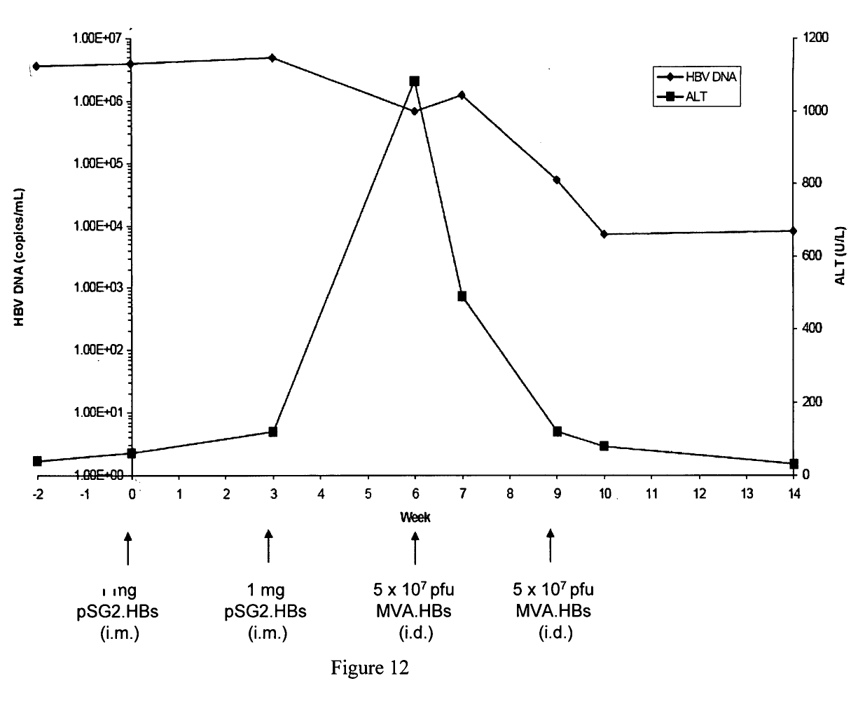

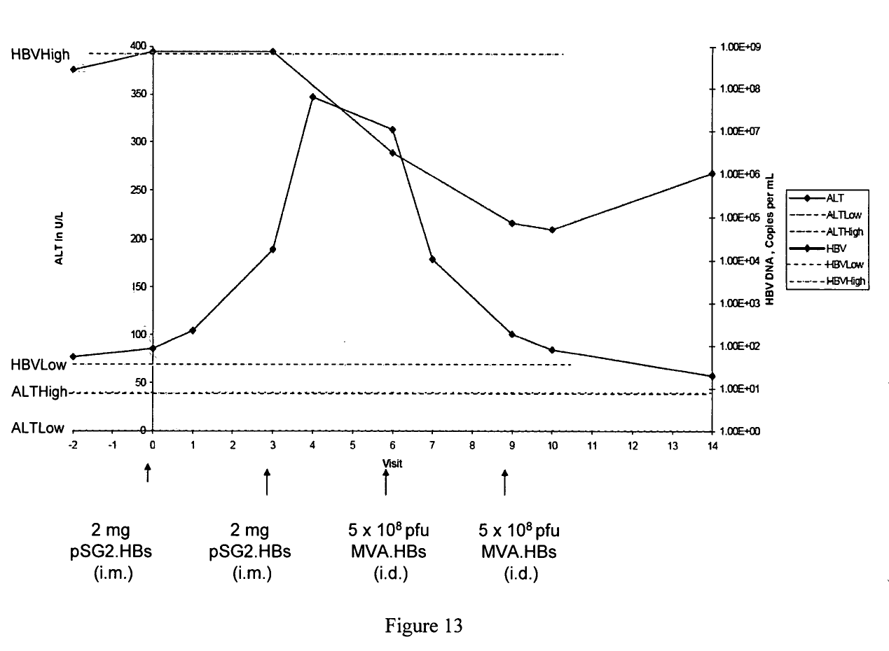

Two treated subjects, subject 102 from Group 1 (see Example 5 and Table 11) and subject 421 from Group A (see Example 6 and Table 12), experienced an alanine transferase (ALT) activity “flare” (i.e., a significant increase in ALT activity) at approximately the same time following the second injection of pSG2.HBs plasmid DNA during their respective treatment regimens (FIGS. 12 and 13). The flares resulted in very high levels of ALT activity, which required both patients to be hospitalised for observation. Therefore, the detection of the flares were recorded as serious adverse events (SAEs), described in Example 5.

Increases in ALT activity appear to have been induced following the first (subject 421) or second (subject 102) injection of pSG2.HBs DNA. In both patients, ALT activity levels peaked and returned to baseline levels shortly thereafter. ALT flares are often correlated with viral clearance, and both subjects 102 and 421 demonstrated seroconversion by week 14 (see Tables 11 and 12, respectively). FIGS. 12 and 13 also indicate that subjects 102 and 421 showed a significant loss of viral load by week 14, coinciding with the timing of the ALT flare. Therefore, in at least two subjects, immune responses against HBV appear to have been induced by injection of pSG2.HBs plasmid DNA, prior to injection of MVA.HBs. This is a surprising result, as injection of DNA alone has been generally considered to be poorly immunogenic.

### EXAMPLE 8

### Analysis of Memory T Cell Immune Responses by In Vitro Stimulation (IVS) IFN-γ ELISPOT

Ex vivo IFN-γ ELISPOT analysis provides a measure of the effector T cell immune response against a specific antigen. The effector T cell immune responses determined by ex vivo IFN-γ ELISPOT against the HBsAg in patients participating in Parts 1 and 2 of the clinical trial (Examples 5 and 6), although satisfying the endpoint of mean +/− s.d. of the response to a peptide pool being greater than mean +3 s.d. of a negative control, were of very low magnitude and more prevalent in patients that received treatment with lamivudine (Part 2, Groups B and C) than in patients who received heterologous PrimeBoost alone (Part 2, Group A). As shown in Table 12, only 6/11 of the Group A patients showed an effector T cell immune response.

Recent clinical studies discussed in Keating et al., (Journal of Immunology 2005, 175:5675-5680) have suggested that ex vivo ELISPOT responses to heterologous PrimeBoost peak at 7 days, and begin falling 14 days, after the boosting vaccination. This may be due to the contraction of the effector T cell population, leaving behind a memory T cell population, which is able to proliferate and respond to future antigen challenge. The memory T cells detected by IVS ELISPOT may be evidence of a more durable, long-term immune response induced by heterologous Prime-Boost.

### Materials and Methods

### In Vitro Stimulation (IVS)

PBMC were thawed and resuspended in RN 10 media (RPMI 1640 media (Invitrogen), supplemented with 2mM L-glutamine, 50 units/mL penicillin, 50 μg/mL streptomycin and 10% heat inactivated foetal bovine serum) to a final concentration between 4-5×10⁶ cells/mL.

PBMC were incubated with a combined pool of all the Hepatitis B peptides described in Example 6, at a final peptide concentration of 2.5 μg/peptide/mL for 14 days. At days 3 and 7 of incubation, the cells were stimulated by addition of 20 units of IL-2 for 4 days each.

### ELISPOT Assays

Microtitre plates were coated with anti-IFN-gamma capture antibody (MAb 1-D1k). 1×10⁵ PBMC were added to each well and incubated overnight with one of the four HBs peptide pools described in Example 6.

Following incubation, PBMC were removed and any secreted IFN-gamma was detected following incubation with a biotinylated anti-IFN detecting antibody (mAb 7-B6-1) followed by a streptavidin-alkaline phosphatase conjugate and subsequent colour development with BCIP (5-bromo-4-chloro-3-indolul phosphate) and NBT (nitroblu tetrazolium). The spots in each well were counted with an AID ELISpot reader, running ELISpot Version 3.1. The number of spots from the four HBs peptide pools were summed and the total was expressed as the number of IFN-gamma secreting cells (spot-forming cells; SFC) per million splenocytes.

### Results

After analysis by in vitro stimulation (IVS) followed by IFN-γ ELISPOT assay, samples from the same patients showed a different pattern of responses to the ex vivo ELISPOT assay. All patients tested from Part 2 Group A (vaccine alone) of the study (Patients 405, 407, 408, 416, 419) showed an increased IFN-γ ELISPOT response at week 10 and/or week 14 (FIG. 14A). Thus, all patients in this group showed an increased response after the administration of heterologous PrimeBoost with pSG2.HBs and MVA.HBs. In contrast, none of the patients tested from Group C (Patients 605 and 607) (FIG. 14C) showed an increase in the IFN-γ ELISPOT responses during the time course, indicating that there was no marked effect after anti-viral treatment with Lamivudine alone. Interestingly, patients from Group B, who received both Lamivudine treatment and heterologous PrimeBoost, exhibited a mixture of responses (FIG. 14B) that were similar to those observed in Group A (Patients 509 and 517) and those in Group C (Patients 510 and 519).

Preliminary studies have indicated that the IFN-γ ELISPOT responses observed include contributions from both CD4+ and CD8+ T cells (data not shown).

In the IVS assay, PBMC are cultured for 14 days in the presence of HBsAg antigen-derived overlapping peptides and IL-2 before the T cell immune response is determined by IFN-γ ELISPOT. Thus, IVS assays provide a measure of antigen-specific memory T cells that proliferate on interaction with their target-antigen. Thus, the IVS assay using HBsAg provides an indication of the memory T cell immune response elicited in clinical samples. By conducting IVS using the HBsAg antigen on patient samples from clinical trial OP02/IVB/001, we have demonstrated that PrimeBoost treatment induces memory T cell immune responses against the HBsAg. These responses were evident in all patients that were tested from Group A, which received PrimeBoost immunization only. Notably, this group also exhibited the highest level of seroconversion against HBeAg, but showed the lowest magnitude of immune responses against HBsAg in the ex vivo IFN-γ ELISPOT assays. Considered together, these results indicate that heterologous PrimeBoost immunization elicited memory T cell responses against the HBsAg, and increased clinical effect against the disease.

All references cited herein are incorporated by reference in their entirety.

While this invention has been particularly shown and described with references to preferred embodiments thereof, it will be understood by those skilled in the art that various changes in form and details may be made therein without departing from the scope of the invention encompassed by the appended claims.

What is claimed is:

1. An isolated nucleic acid molecule comprising a nucleotide sequence that encodes SEQ ID NO:3, or a fragment thereof.

2. A recombinant DNA plasmid or a non-replicating or replication impaired poxvirus comprising the nucleic acid molecule of claim 1, wherein the nucleic acid molecule comprises SEQ ID NO:1.

3. A pharmaceutical composition comprising a nucleic acid molecule according to claim 1.

4. A method of inducing an immune response against a hepatitis B antigen in a mammal comprising administering to the mammal a nucleic acid molecule according to claim 1.

5. The method of claim 4, wherein a de novo immune response against the hepatitis B antigen is induced, a preexisting immune response against hepatitis B is boosted, or seroconversion of one or more HBV antigens is induced, in the mammal.

6. The method of claim 4, wherein the nucleic acid molecule is administered in multiple doses.

7. The method of claim 6, wherein the nucleic acid molecule is administered twice.

8. An isolated recombinant replication-deficient poxvirus comprising the nucleotide sequence of SEQ ID NO:4 or SEQ ID NO:5.

9. The poxvirus according to claim 8, wherein the poxvirus is a modified vaccinia virus Ankara.

10. A pharmaceutical composition comprising the poxvirus according to claim 8.

11. A method of inducing an immune response against a hepatitis B antigen in a mammal, comprising administering to the mammal the poxvirus of claim 8.

12. The method of claim 11, wherein the poxvirus is administered to the mammal following administration of a composition comprising a nucleotide sequence that encodes SEQ ID NO:3, or a fragment thereof.

13. A priming composition and a boosting composition for sequential administration to an individual to treat hepatitis B; wherein
the priming composition comprises the nucleic acid molecule of claim 1; and
the boosting composition comprises a recombinant replication-deficient poxvirus comprising the nucleotide sequence of SEQ ID NO:4 or SEQ ID NO:5.

14. A method of inducing an immune response against a hepatitis B antigen in an individual, comprising administering to the individual the priming and boosting compositions of claim 13.

15. A method of inducing an immune response against hepatitis B in a subject comprising:
a) administering to the subject a priming composition comprising a DNA plasmid comprising SEQ ID NO:4 or SEQ ID NO:5; followed by
b) administering to the subject a boosting composition comprising a recombinant MVA vector comprising SEQ ID NO:4 or SEQ ID NO:5.

16. The method of claim 15, wherein the immune response comprises a memory T cell response selected from the group consisting of a CD8⁺ memory T cell response and a CD4⁺ memory T cell response.

17. A kit for inducing an immune response against hepatitis B in a subject, comprising:
a) a priming composition comprising a DNA plasmid comprising SEQ ID NO:4 or SEQ ID NO:5; and
b) a boosting composition comprising the recombinant MVA of claim 9.

18. A kit comprising:
(i) a priming composition comprising a nucleic acid molecule that encodes SEQ ID NO:3, or a fragment thereof;
(ii) a boosting composition comprising the poxvirus of claim 8; and
(iii) instructions to administer the priming composition one or more times followed by the boosting composition one or more times to an individual.

19. The kit of claim 17, comprising:
(i) two doses of the priming composition;
(ii) two doses of the boosting composition; and
(iii) instructions to administer the priming composition twice followed by the boosting composition twice.

20. The kit of claim 18, comprising:
(i) two doses of the priming composition;
(ii) two doses of the boosting composition; and
(iii) instructions to administer the priming composition twice followed by the boosting composition twice.
